(19) United States
(12) Patent Application Publication
(10) Pub. No.: US 2012/0301415 A1
(43) Pub. Date: Nov. 29, 2012

(54) COMPOSITIONS HAVING ENHANCED COSMETIC PROPERTIES

(75) Inventors: Hy Si BUI, Piscataway, NJ (US); Wei Hong YU, Edison, NJ (US); Shao Xiang LU, Shanghai (CN); Florentina PAVEL, Hillsborough, NJ (US); Susan HALPERN, Basking Ridge, NJ (US)

(73) Assignee: L'OREAL, Paris (FR)

(21) Appl. No.: 13/571,499

(22) Filed: Aug. 10, 2012

Related U.S. Application Data
(63) Continuation of application No. 11/584,994, filed on Oct. 23, 2006, now abandoned.
(60) Provisional application No. 60/729,504, filed on Oct. 24, 2005.

(51) Int. Cl. A61K 8/58; A61Q 1/06; A61Q 1/10; A61Q 1/02

(52) U.S. Cl. 424/64; 424/63; 424/70.7; 424/70.121

(57) ABSTRACT

A cosmetic composition and process which provides enhanced wear, comfort and feel, the composition containing: (a) at least one polypropylsilsesquioxane film forming resin; (b) at least one polymer chosen from a polyorganosiloxane copolymer and a silicone acrylate copolymer; (c) at least one solvent; and (d) optionally, at least one colorant.

### CROSS REFERENCE TO RELATED APPLICATION

This application claims the benefit of the filing date of U.S. Provisional Application No. 60/729,504 filed Oct. 24, 2005, and U.S. Non-Provisional application Ser. No. 11/584,994 filed Oct. 23, 2006, the contents of which are incorporated by reference.

### BACKGROUND OF THE INVENTION

The present invention relates to a cosmetic composition, especially a makeup and/or care composition for keratinous materials comprising a silicone resin in a cosmetically acceptable medium. The invention also relates to a makeup process or care process for the keratinous materials.

The use of film-forming silicone resins in cosmetic compositions has been described, for example, in U.S. Pat. No. 5,505,937, U.S. Pat. No. 5,911,974, U.S. Pat. No. 5,965,112, U.S. Pat. No. 5,985,298, U.S. Pat. No. 6,074,654, US 20020031488, U.S. Pat. No. 6,780,422, US 20040156806. These patents and patent applications describe and claim compositions containing various silicone resins in association with several specific ingredients. The presence of these silicone resins makes it possible to increase the wear of the composition applied to the skin, lips or lashes. However, while these cosmetic compositions are drying on the skin, they often give rise to sensations of tautness that the user finds unpleasant, making the composition uncomfortable to wear. These problems of comfort are especially associated with the mechanical properties of the deposits obtained on the skin. In particular, when the film formed on the skin after applying the composition is too rigid, it leaves an unpleasant sensation, for example during movements of the face.

Although the use of silicone resins, in general, does increase the wear of cosmetic compositions due to their ability to form films, the rigidity of the film may cause it to flake off from the keratinous material onto which it is applied, thereby reducing its wear properties.

There is thus still a need for a stable cosmetic composition to be applied to the skin, which forms a film that is both comfortable on the skin and shows good wear properties in terms of its resistance to flaking.

Surprisingly, the Applicant has found that the combination of a polypropylsilsesquioxane film forming resin with at least one polymer chosen from a polyorganosiloxane copolymer and a silicone acrylate copolymer, in a cosmetically acceptable medium gives a deposit on the skin that shows noteworthy cosmetic properties. In particular, such a composition is comfortable to apply, provides a cushiony feel and shows good wear properties. The use of this polypropylsilsesquioxane film forming resin can also obviate the use of a plasticizer.

### BRIEF SUMMARY OF THE INVENTION

The present invention relates to a cosmetic composition containing:

a) at least one polypropylsilsesquioxane film forming resin;

b) at least one polymer chosen from a polyorganosiloxane copolymer and a silicone acrylate copolymer;

c) at least one solvent; and

d) optionally, at least one colorant.

The invention also relates to a process for treating a keratinous material involving contacting the keratinous material with a cosmetic composition containing:

a) at least one polypropylsilsesquioxane film forming resin;

b) at least one polymer chosen from a polyorganosiloxane copolymer and a silicone acrylate copolymer;

c) at least one solvent; and

d) optionally, at least one colorant.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

It has been unexpectedly discovered that a cosmetic composition containing at least one polypropylsilsesquioxane film forming resin and a polyorganosiloxane and/or a silicone acrylate copolymer provides enhanced wear properties.

Without intending to be bound by theory, it is believed that the propyl substituted polymeric silsesquioxane film forming resin in combination with the polyorganosiloxane and/or silicone acrylate copolymer, utilized in a cosmetically acceptable composition, provides a more flexible, homogeneous, and less rigid film on the keratinous material resulting in comfortable, and extended wear.

The invention may be used as an eyeshadow, an eyeliner, a blush, a foundation, a mascara, or a lip composition. In addition, the product of the invention, instead of being molded in the form of a stick, may also be hot-poured.

The term “keratinous material” is meant to include hair and skin.

By the term “resin” it is meant that the polypropylsilsesquioxane film forming polymer provides substantive, film forming properties when applied to skin.

The term “film forming” means that the polypropylsilsesquioxane polymer is capable of forming a film, in particular, a substantive film, on the keratinous surface to which it is applied.

Moreover, by “comprising” it is meant that other steps and/or ingredients which do not affect the end result may be added. The products, compositions, methods and processes of the present invention can include all the essential elements and limitations of the invention described herein as well as any of the additional or optional ingredients, components, steps, or limitations described herein.

All percentages, parts and ratios are based upon the total weight of the compositions of the present invention unless otherwise indicated.

The compositions and processes, including the essential and additional or optional components, are described in detail below.

Polypropylsilsesquioxane Film-Forming Resin

Silsesquioxane resins are a specific form of silicone resins. Silicone resin nomenclature is known in the art as “MDTQ” nomenclature, whereby a silicone resin is described according to the various monomeric siloxane units which make up the polymer.

Each letter of “MDTQ” denotes a different type of unit. The letter M denotes the monofunctional unit (CH₃)₃SiO_{1/2}. This unit is considered to be monofunctional because the silicone atom only shares one oxygen when the unit is part of a polymer. The “M” unit can be represented by the following structure:

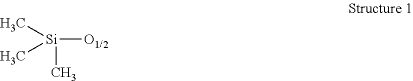

At least one of the methyl groups of the M unit may be replaced by another group, e.g., to give a unit with formula [R(CH₃)₂]SiO_{1/2}, as represented in the following structure:

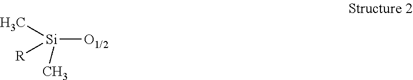

wherein R is chosen from groups other than methyl groups. Non-limiting examples of such groups other than methyl groups include alkyl groups other than methyl groups, alkene groups, alkyne groups, hydroxyl groups, thiol groups, ester groups, acid groups, ether groups, wherein the groups other than methyl groups may be further substituted.

The symbol D denotes the difunctional unit (CH₃)₂SiO_{2/2} wherein two oxygen atoms bonded to the silicone atom are used for binding to the rest of the polymer. The “D” unit, which is the major building block of dimethicone oils, can be represented as:

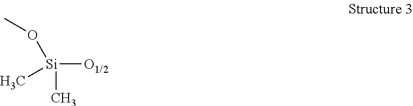

Here again, at least one of the methyl groups of the D unit may be replaced by another group, e.g., to give a unit with formula RₐR_{b}SiO_{2/2}, RCH₃ SiO_{2/2} wherein Rₐ and R_{b} may be chosen from groups other than methyl groups. Non-limiting examples of such groups other than methyl groups include alkyl groups other than methyl groups, alkene groups, alkyne groups, hydroxyl groups, thiol groups, ester groups, acid groups, ether groups, wherein the groups other than methyl groups may be further substituted.

The symbol T denotes the trifunctional unit, CH₃SiO_{3/2} and can be represented as:

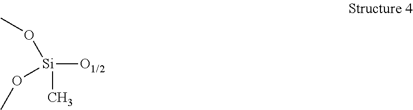

Here again, the methyl group may be replaced by another group, e.g., to give a unit with formula RSiO_{3/2}, wherein R may be chosen from groups other than methyl groups. Non-limiting examples of such groups other than methyl groups include alkyl groups other than methyl groups, alkene groups, alkyne groups, hydroxyl groups, thiol groups, ester groups, acid groups, ether groups, phenyl groups, alkoxy groups, wherein the groups other than methyl groups may be further substituted.

Similarly, the symbol Q denotes the tetrafunctional unit, SiO_{4/2} wherein all four oxygens bonded to the silicone atom are bonded to the rest of the polymer.

When the film forming resin is made up predominantly of trifunctional units or T units, it is generally called a silsesquioxane resin. If it is made up primarily of repeating units as depicted in Structure 4, it is called a polymethylsilsesquioxane. A non-limiting example of the at least one polymethylsilsesquioxane film former is Belsil PMS MK, also referred to as Resin MK, available from Wacker Chemie. This polymethylsilsesquioxane film former is a polymer comprising polymerized repeating units of CH₃SiO_{3/2} (T units) and may also contain up to 1% by weight or by mole of units of the formula (CH₃)₂SiO_{2/2} (D units). The weight-average molecular weight of this polymer has been estimated to be 10,000.

When the film forming resin is made up predominantly of trifunctional RSiO_{3/2} units or T units, wherein R is a propyl radical, it is called a polypropylsilsesquioxane.

One example of a polypropylsilsesquioxane resin suitable for use in the present invention is commercially available from Dow-Corning as Dow Corning 670 Fluid. Dow Corning 670 Fluid has a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO_{3/2} units, and having a hydroxyl or alkoxy content from 0.2 to 10% by weight, for example between 1 and 4% by weight, preferably between 5 and 10% by weight, and more preferably between 6 and 8% by weight.

The film forming polypropylsilsesquioxane resin may be present in an amount ranging from 0.5 to 80% by weight, preferably from 5 to 70% by weight, more preferably from 10 to 50% by weight, more preferably from 15 to 40% by weight, and more preferably from 20 to 30% by weight, based on the weight of the total composition.

The Polyorganosiloxane Copolymer

Polymers of the polyorganosiloxane type are described in documents U.S. Pat. No. 5,874,069, U.S. Pat. No. 5,919,441, U.S. Pat. No. 6,051,216 and U.S. Pat. No. 5,981,680, the entire contents of which are hereby incorporated by reference.

These polymers may belong to the following two families:

a) polyorganosiloxanes comprising at least two groups capable of establishing hydrogen interactions, these two groups being located in the polymer chain; and/or

b) polyorganosiloxanes comprising at least two groups capable of establishing hydrogen interactions, these two groups being located on grafts or branches.

They are solids that may be dissolved beforehand in a solvent with hydrogen interaction capable of breaking the hydrogen interactions of the polymers, for instance C₂ to C₈ lower alcohols and especially ethanol, n-propanol or isopropanol, before being placed in the presence of the silicone oils according to the invention. It is also possible to use these hydrogen interaction “breaking” solvents as co-solvents. These solvents may then be stored in the composition or may be removed by selective evaporation, which is well known to those skilled in the art.

The polymers comprising two groups capable of establishing hydrogen interactions in the polymer chain may be polymers comprising at least one moiety corresponding to the formula:

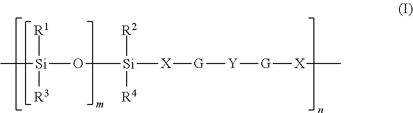

in which:

1) R¹, R², R³ and R⁴, which may be identical or different, represent a group chosen from:
linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulphur and/or nitrogen atoms;

2) the groups X, which may be identical or different, represent a linear or branched C₁ to C₂₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or

4) Y represents a group corresponding to the formula:

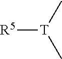

in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁵ represents a linear or branched C₁ to C₅₀ alkyl group or a polyorganosiloxane chain, possibly comprising one or more ester, amide, urethane, thiocarbamate, urea, thiourea and/or sulphonamide groups, which may be linked to another chain of the polymer;

5) the groups G, which may be identical or different, represent divalent groups chosen from:

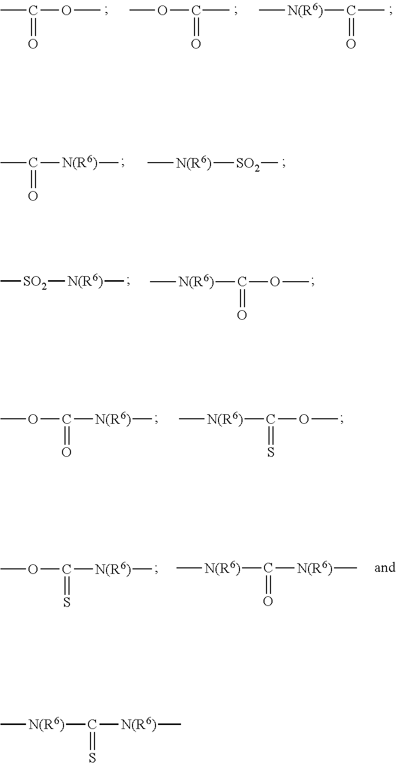

in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than:

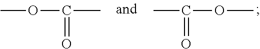

6) n is an integer ranging from 2 to 500 and preferably from 2 to 200, and m is an integer ranging from 1 to 1000, preferably from 1 to 700 and better still from 6 to 200.

According to the invention, 80% of the groups R¹, R², R³ and R⁴ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other moieties of the polymer or copolymer. Preferably, Y represents a group chosen from:

a) linear C₁ to C₂₀ and preferably C₁ to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,

g) polyorganosiloxane chains of formula:

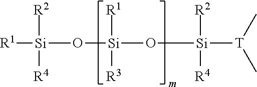

in which R¹, R², R³, R⁴, T and m are as defined above, and

h) polyorganosiloxane chains of formula:

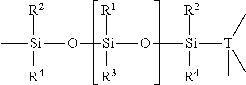

The polyorganosiloxanes of the second family may be polymers comprising at least one moiety corresponding to formula (II):

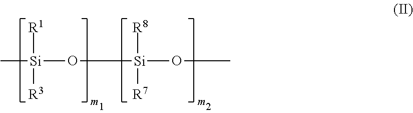

in which
R¹ and R³, which may be identical or different, are as defined above for formula (I),
R⁷ represents a group as defined above for R¹ and R³, or represents a group of formula —X-G-R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R⁸ represents a group of formula —X-G-R⁹ in which X, G and R⁹ are as defined above,
m₁ is an integer ranging from 1 to 998, and
m₂ is an integer ranging from 2 to 500.

According to the invention, the polymer used as gelling agent may be a homopolymer, that is to say a polymer comprising several identical moieties, in particular moieties of formula (I) or of formula (II).

According to the invention, it is also possible to use a polymer consisting of a copolymer comprising several different moieties of formula (I), that is to say a polymer in which at least one of the groups R¹, R², R³, R⁴, X, G, Y, m and n is different in one of the moieties. The copolymer may also be formed from several moieties of formula (II), in which at least one of the groups R¹, R³, R⁷, R⁸, m₁ and m₂ is different in at least one of the moieties.

It is also possible to use a copolymer comprising at least one moiety of formula (I) and at least one moiety of formula (II), the moieties of formula (I) and the moieties of formula (II) possibly being identical to or different from each other.

According to one variant of the invention, it is also possible to use a copolymer furthermore comprising at least one hydrocarbon-based moiety comprising two groups capable of establishing hydrogen interactions, chosen from ester, amide, sulphonamide, carbamate, thiocarbamate, urea and thiourea groups, and combinations thereof.

These copolymers may be block copolymers or grafted copolymers.

According to one embodiment of the invention, the groups capable of establishing hydrogen interactions are amide groups of formulae —C(O)NH— and —HN—C(O)—.

In this case, the polyorganosiloxane may be a polymer comprising at least one moiety of formula (III) or (IV):

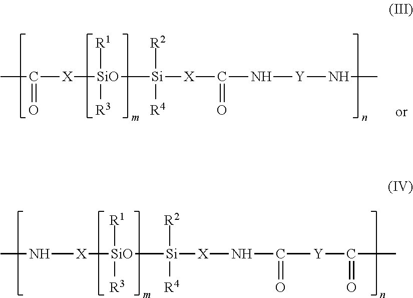

in which R¹, R², R³, R⁴, X, Y, m and n are as defined above.

Such a moiety may be obtained:
either by a condensation reaction between a silicone containing α, ω-carboxylic acid ends and one or more diamines, according to the following reaction scheme:

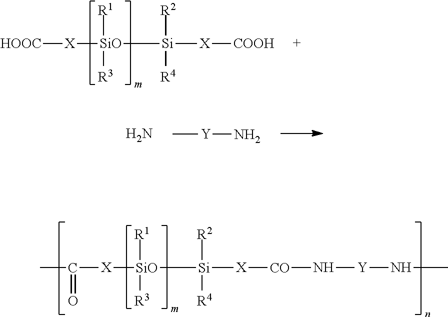

or by reaction of two molecules of α-unsaturated carboxylic acid with a diamine according to the following reaction scheme:

CH₂═CH—X¹—COOH+H₂N—Y—NH₂→CH₂═CH—X¹—CO—NH—Y—NH—CO—X¹—CH═CH₂

followed by the addition of a siloxane to the ethylenic unsaturations, according to the following scheme:

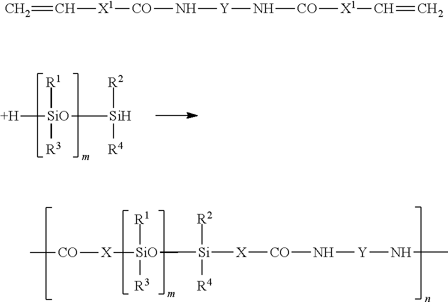

in which X¹—(CH₂)₂— corresponds to X defined above and Y, R¹, R², R³, R⁴ and m are as defined above;
or by reaction of a silicone containing α,ω-NH₂ ends and a diacid of formula HOOC—Y—COOH according to the following reaction scheme:

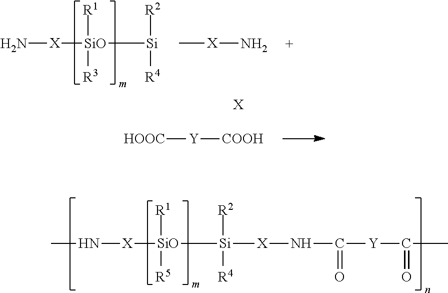

In these polyamides of formula (III) or (IV), m is preferably in the range from 1 to 700, more preferably from 15 to 500 and better still from 15 to 45, and n is in particular in the range from 1 to 500, preferably from 1 to 100 and better still from 4 to 25, X is preferably a linear or branched alkylene chain containing from 1 to 30 carbon atoms and in particular 3 to 10 carbon atoms, and Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.

In formulae (III) and (IV), the alkylene group representing X or Y can optionally contain in its alkylene portion at least one of the following elements:

1) 1 to 5 amide, urea or carbamate groups,

2) a C₅ or C₆ cycloalkyl group, and

3) a phenylene group optionally substituted with 1 to 3 identical or different C₁ to C₃ alkyl groups.

In formulae (III) and (IV), the alkylene groups may also be substituted with at least one element chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

In these formulae (III) and (IV), Y may also represent:

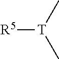

in which R⁵ represents a polyorganosiloxane chain and T represents a group of formula:

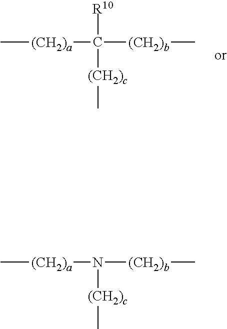

in which a, b and c are, independently, integers ranging from 1 to 10, and R¹⁰ is a hydrogen atom or a group such as those defined for R¹, R², R³ and R⁴.

In formulae (III) and (IV), R¹, R², R³ and R⁴ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different moieties of formula (III) or (IV).

Thus, the polymer may be a polyamide containing several moieties of formula (III) or (IV) of different lengths, i.e. a polyamide corresponding to the formula:

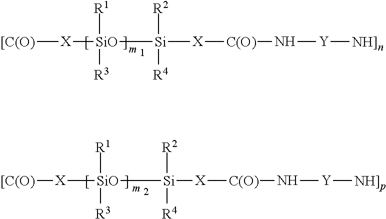

in which X, Y, n and R¹ to R⁴ have the meanings given above, m₁ and m₂, which are different, are chosen in the range from 1 to 1,000, and p is an integer ranging from 2 to 300.

In this formula, the moieties may be structured to form either a block copolymer, or a random copolymer or an alternating copolymer. In this copolymer, the moieties may be not only of different lengths, but also of different chemical structures, for example containing different groups Y. In this case, the copolymer may correspond to the formula:

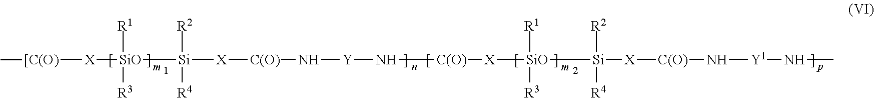

in which R¹ to R⁴, X, Y, m₁, m₂, n and p have the meanings given above and Y¹ is different from Y but chosen from the groups defined for Y. As previously, the various moieties may be structured to form either a block copolymer, or a random copolymer or an alternating copolymer.

In another embodiment of the invention, the polyorganosiloxane may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one moiety of formula:

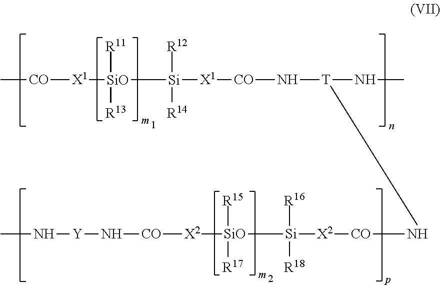

in which X¹ and X², which may be identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

In formula (VII), it is preferred that:
p is in the range from 1 to 25 and better still from 1 to 7,
R¹¹ to R¹⁸ are methyl groups,
T corresponds to one of the following formulae:

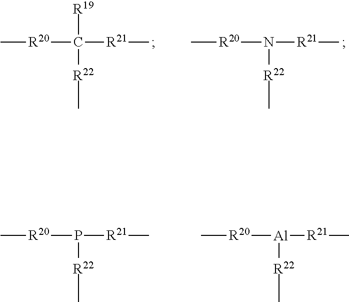

in which R¹⁹ is a hydrogen atom or a group chosen from the groups defined for R¹ to R⁴, and R²⁰, R²¹ and R²² are, independently, linear or branched alkylene groups, and more preferably corresponds to the formula:

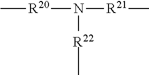

in particular with R²⁰, R²¹ and R²² representing —CH₂—CH₂—,
m₁ and m₂ are in the range from 15 to 500 and better still from 15 to 45,
X¹ and X² represent —(CH₂)₁₀—, and
Y represents —CH₂—.

These polyamides containing a grafted silicone moiety of formula (VII) may be copolymerized with polyamide-silicones of formula (II) to form block copolymers, alternating copolymers or random copolymers. The weight percentage of grafted silicone moieties (VII) in the copolymer may range from 0.5% to 30% by weight.

According to the invention, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to the invention, the preferred siloxane-based polyamides are:
polyamides of formula (III) in which m is from 15 to 50;
mixtures of two or more polyamides in which at least one polyamide has a value of m in the range from 15 to 50 and at least one polyamide has a value of m in the range from 30 to 50;
polymers of formula (V) with m₁ chosen in the range from 15 to 50 and m₂ chosen in the range from 30 to 500 with the portion corresponding to m₁ representing 1% to 99% by weight of the total weight of the polyamide and the corresponding portion m₂ representing 1% to 99% by weight of the total weight of the polyamide;
mixtures of polyamide of formula (III) combining:

1) 80% to 99% by weight of a polyamide in which n is equal to 2 to 10 and in particular 3 to 6, and

2) 1% to 20% of a polyamide in which n is in the range from 5 to 500 and in particular from 6 to 100;
polyamides corresponding to formula (VI) in which at least one of the groups Y and Y¹ contains at least one hydroxyl substituent;
polyamides of formula (III) synthesized with at least one portion of an activated diacid (diacid chloride, dianhydride or diester) instead of the diacid;
polyamides of formula (III) in which X represents —(CH₂)₃— or —(CH₂)₁₀; and
polyamides of formula (III) in which the polyamides end with a monofunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

According to the invention, the end groups of the polymer chain may end with:
a C₁ to C₅₀ alkyl ester group by introducing a C₁ to C₅₀ monoalcohol during the synthesis,
a C₁ to C₅₀ alkylamide group by taking as stopping group a monoacid if the silicone is α,ω-diaminated, or a monoamine if the silicone is an α,ω-dicarboxylic acid.

According to one embodiment variant of the invention, it is possible to use a copolymer of silicone polyamide and of hydrocarbon-based polyamide, i.e. a copolymer comprising moieties of formula (III) or (IV) and hydrocarbon-based polyamide moieties. In this case, the polyamide-silicone moieties may be arranged at the ends of the hydrocarbon-based polyamide.

Polyamide-based polyorganosiloxanes may be produced by silylic amidation of polyamides based on fatty acid dimer. This approach involves the reaction of free acid sites existing on a polyamide as end sites, with organosiloxane-monoamines and/or organosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups, may be used for the reaction with hydrocarbon-based polyamides based on fatty acid dimers. Siloxane diamines containing 13.5 siloxane groups are preferred, and the best results are obtained with the siloxane diamine containing 13.5 siloxane groups and polyamides containing high numbers of carboxylic acid end groups.

The reactions may be carried out in xylene to extract the water produced from the solution by azeotropic distillation, or at higher temperatures (about 180 to 200° C.) without solvent. Typically, the efficacy of the amidation and the reaction rates decrease when the siloxane diamine is longer, that is to say when the number of siloxane groups is higher. Free amine sites may be blocked after the initial amidation reaction of the diaminosiloxanes by reacting them either with a siloxane acid, or with an organic acid such as benzoic acid.

For the esterification of the free acid sites on the polyamides, this may be performed in boiling xylene with about 1% by weight, relative to the total weight of the reagents, of para-toluenesulphonic acid as catalyst.

These reactions carried out on the carboxylic acid end groups of the polyamide lead to the incorporation of silicone moieties only at the ends of the polymer chain.

It is also possible to prepare a copolymer of polyamide-silicone, using a polyamide containing free amine groups, by amidation reaction with a siloxane containing an acid group.

It is also possible to prepare a polyorganosiloxane based on a copolymer between a hydrocarbon-based polyamide and a silicone polyamide, by transamidation of a polyamide having, for example, an ethylene-diamine constituent, with an oligosiloxane-α,ω-diamine, at high temperature (for example 200 to 300° C.), to carry out a transamidation such that the ethylenediamine component of the original polyamide is replaced with the oligosiloxane diamine.

The copolymer of hydrocarbon-based polyamide and of polyamide-silicone may also be a grafted copolymer comprising a hydrocarbon-based polyamide backbone with pendent oligosiloxane groups.

This may be obtained, for example:
by hydrosilylation of unsaturated bonds in polyamides based on fatty acid dimers;
by silylation of the amide groups of a polyamide; or
by silylation of unsaturated polyamides by means of an oxidation, that is to say by oxidizing the unsaturated groups into alcohols or diols, to form hydroxyl groups that are reacted with siloxane carboxylic acids or siloxane alcohols. The olefinic sites of the unsaturated polyamides may also be epoxidized and the epoxy groups may then be reacted with siloxane amines or siloxane alcohols.

According to another embodiment of the invention, the polyorganosiloxane consists of a homopolymer or a copolymer comprising urethane or urea groups.

As previously, the polymer may comprise polyorganosiloxane moieties containing two or more urethane and/or urea groups, either in the backbone of the polymer or on side chains or as pendent groups.

The polymers comprising at least two urethane and/or urea groups in the backbone may be polymers comprising at least one moiety corresponding to the following formula:

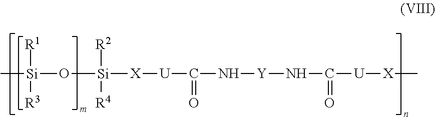

in which R¹, R², R³, R⁴, X, Y, m and n have the meanings given above for formula (I), and U represents —O— or —NH—, such that:

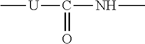

corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. Preferably, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to the formula:

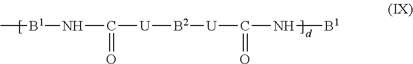

in which B¹ is a group chosen from the groups given above for Y, U is —O— or —NH— and B² is chosen from:

linear or branched C₁ to C₄₀ alkylene groups, which can optionally bear an ionizable group such as a carboxylic acid or sulphonic acid group, or a neutralizable or quaternizable tertiary amine group;

C₅ to C₁₂ cycloalkylene groups, optionally bearing alkyl substituents, for example one to three methyl or ethyl groups, or alkylene, for example the diol radical: cyclohexanedimethanol;

phenylene groups that may optionally bear C₁ to C₃ alkyl substituents; and

groups of formula:

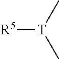

in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.

T can represent, for example:

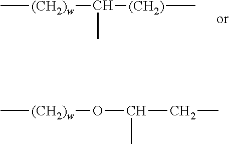

with w being an integer ranging from 1 to 10 and R⁵ being a polyorganosiloxane chain.

When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C₁ to C₄₀ alkylene group, in particular —(CH₂)₂— or —(CH₂)₆— or a group:

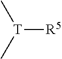

with R⁵ being a polyorganosiloxane chain.

As previously discussed, the polyorganosiloxane may be formed from silicone urethane and/or silicone urea moieties of different length and/or constitution, and may be in the form of block or random copolymers.

According to the invention, the silicone may also comprise urethane and/or urea groups no longer in the backbone but as side branches.

In this case, the polymer may comprise at least one moiety of formula:

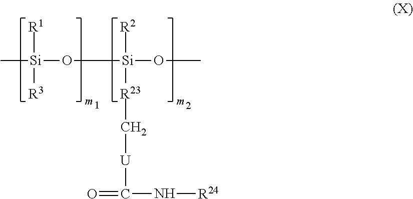

in which R¹, R², R³ m₁ and m₂ have the meanings given above for formula (I),
U represents O or NH,
R²³ represents a C₁ to C₄₀ alkylene group, optionally comprising one or more hetero atoms chosen from O and N, or a phenylene group, and
R²⁴ is chosen from linear, branched or cyclic, saturated or unsaturated C₁ to C₅₀ alkyl groups, and phenyl groups optionally substituted with one to three C₁ to C₃ alkyl groups.

The polymers comprising at least one moiety of formula (X) contain siloxane units and urea or urethane groups, and they may be used as gelling agents in the compositions of the invention.

The siloxane polymers may have a single urea or urethane group by branching or may have branches containing two urea or urethane groups, or alternatively they may contain a mixture of branches containing one urea or urethane group and branches containing two urea or urethane groups.

They may be obtained from branched polysiloxanes, comprising one or two amino groups by branching, by reacting these polysiloxanes with monoisocyanates.

As examples of starting polymers of this type containing amino and diamino branches, mention may be made of the polymers corresponding to the following formulae:

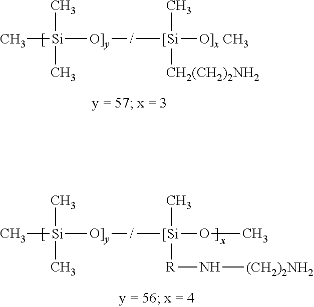

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branching may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polyorganosiloxanes that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula:

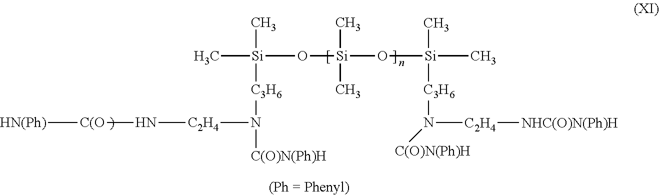

in which Ph is a phenyl group and n is a number from 0 to 300, in particular from 0 to 100, for example 50.

This polymer is obtained by reacting the following polysiloxane containing amino groups:

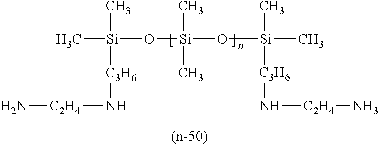

with phenyl isocyanate.

The polymers of formula (VIII) comprising urea or urethane groups in the chain of the silicone polymer may be obtained by reaction between a silicone containing α,ω-NH₂ or —OH end groups, of formula:

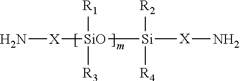

in which m, R¹, R², R³, R⁴ and X are as defined for formula (I) and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoichiometric proportions between the two reagents, diisocyanate and coupling agent, Y may have the formula (IX) with d equal to 0 or d equal to 1 to 5.

As in the case of the polyamide silicones of formula (II) or (III), it is possible to use in the invention polyurethane or polyurea silicones containing moieties of different length and structure, in particular moieties whose lengths differ by the number of silicone units. In this case, the copolymer may correspond, for example, to the formula:

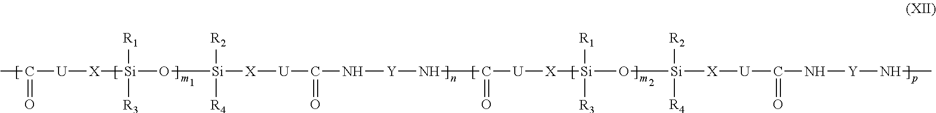

in which R¹, R², R³, R⁴, X, Y and U are as defined for formula (VIII) and m₁, m₂, n and p are as defined for formula (V).

Branched polyurethane or polyurea silicones may also be obtained using, instead of the diisocyanate OCN—Y—NCO, a triisocyanate of formula:

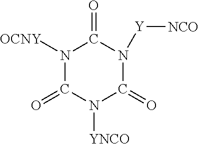

A polyurethane or polyurea silicone containing branches comprising an organosiloxane chain with groups capable of establishing hydrogen interactions is thus obtained. Such a polymer comprises, for example, a moiety corresponding to the formula:

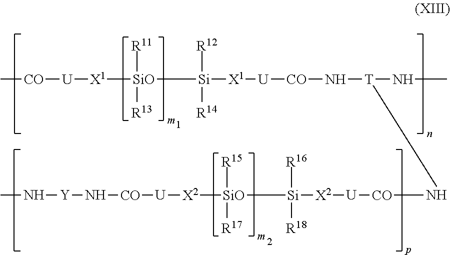

in which X¹ and X², which are identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

As in the case of the polyamides, this copolymer can also comprise polyurethane silicone moieties without branching.

In another embodiment of the invention, the siloxane-based polyureas and polyurethanes that are preferred are:
polymers of formula (VIII) in which m is from 15 to 50;
mixtures of two or more polymers in which at least one polymer has a value of m in the range from 15 to 50 and at least one polymer has a value of m in the range from 30 to 50;
polymers of formula (XII) with m₁ chosen in the range from 15 to 50 and m₂ chosen in the range from 30 to 500 with the portion corresponding to m₁ representing 1% to 99% by weight of the total weight of the polymer and the portion corresponding to m₂ representing 1% to 99% by weight of the total weight of the polymer;
mixtures of polymer of formula (VIII) combining:

1) 80% to 99% by weight of a polymer in which n is equal to 2 to 10 and in particular 3 to 6, and

2) 1% to 20% of a polymer in which n is in the range from 5 to 500 and in particular from 6 to 100;
copolymers comprising two moieties of formula (VIII) in which at least one of the groups Y contains at least one hydroxyl substituent;
polymers of formula (VIII) synthesized with at least one portion of an activated diacid (diacid chloride, dianhydride or diester) instead of the diacid;
polymers of formula (VIII) in which X represents —(CH₂)₃— or —(CH₂)₁₀—; and
polymers of formula (VIII) in which the polymers end with a multifunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

As in the case of the polyamides, copolymers of polyurethane or polyurea silicone and of hydrocarbon-based polyurethane or polyurea may be used in the invention by performing the reaction for synthesizing the polymer in the presence of an α, ω-difunctional block of non-silicone nature, for example a polyester, a polyether or a polyolefin.

As has been seen previously, polyorganosiloxanes consisting of homopolymers or copolymers of the invention may contain siloxane moieties in the main chain of the polymer and groups capable of establishing hydrogen interactions, either in the main chain of the polymer or at the ends thereof, or on side chains or branches of the main chain. This may correspond to the following five arrangements:

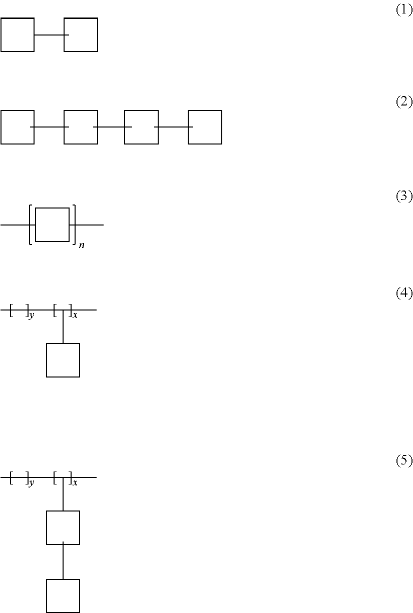

in which the continuous line is the main chain of the siloxane polymer and the squares represent the groups capable of establishing hydrogen interactions.

In case (1), the groups capable of establishing hydrogen interactions are arranged at the ends of the main chain.

In case (2), two groups capable of establishing hydrogen interactions are arranged at each of the ends of the main chain.

In case (3), the groups capable of establishing hydrogen interactions are arranged within the main chain in repeating moieties.

In cases (4) and (5), these are copolymers in which the groups capable of establishing hydrogen interactions are arranged on branches of the main chain of a first series of moieties that are copolymerized with moieties not comprising groups capable of establishing hydrogen interactions. The values n, x and y are such that the polymer has the desired properties in terms of an agent for gelling fatty phases based on silicone oil.

As examples of polymers that may be used, mention may be made of the silicone polyamides obtained in accordance with Examples 1 and 2 of document U.S. Pat. No. 5,981,680.

The polyorganosiloxane copolymers advantageously have a softening point from 20 to 130° C. Preferably, they have a softening point ranging from 65 to 130° C. and better still from 70° C. to 130° C.

The polyorganosiloxane copolymers may be present in an amount ranging from 0.5 to 30% by weight, preferably from 1 to 25% by weight, more preferably from 1 to 20% by weight, more preferably from 1 to 15% by weight, and more preferably from 1 to 10% by weight, based on the weight of the total composition.

The Silicone Acrylate Copolymer

The at least one polymer may also be chosen from silicone/(meth)acrylate copolymers, such as those as described in U.S. Pat. Nos. 5,061,481, 5,219,560, and 5,262,087, the entire contents of which are hereby incorporated by reference. Also included are those derived from non-polar silicone copolymers comprising repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the product sold under the tradename KP-545, or acrylates/stearyl acrylate/dimethicone acrylates copolymers, such as those commercially available from Shin-Etsu, for example, the product sold under the tradename KP-561, and acrylates/behenyl acrylate/dimethicone acrylates copolymer, such as those commercially available from Shin-Etsu, for example, the product sold under the tradename KP-562.

Further non-limiting examples include polymers comprising a backbone chosen from vinyl polymers, methacrylic polymers, and acrylic polymers and at least one chain chosen from pendant siloxane groups and pendant fluorochemical groups. Non-limiting examples of such polymers comprise at least one unit derived from at least one A monomer, at least one unit derived from at least one C monomer, at least one unit derived from D monomers, and, optionally, at least one unit derived from at least one B monomer, wherein:

A, which may be identical or different, are each chosen from free-radically-polymerizable acrylic esters of at least one alcohol chosen from 1,1-dihydroperfluoroalkanols, omega-hydridofluoroalkanols, fluoroalkylsulfonamido alcohols, cyclic fluoroalkyl alcohols, and fluoroether alcohols, and analogs of any of the foregoing at least one alcohols, and free-radically-polymerizable methacrylic esters of at least one alcohol chosen from 1,1-dihydroperfluoroalkanols, omega-hydridofluoroalkanols, fluoroalkylsulfonamido alcohols, cyclic fluoroalkyl alcohols, and fluoroether alcohols, and analogs of any of the foregoing at least one alcohols;

B, which may be identical or different, are each chosen from reinforcing monomers which are copolymerizable with at least one A monomer;

C, which may be identical or different, are each chosen from monomers having the formula:

X(Y)ₙSi(R)₃₋ₘZₘ

wherein

X is chosen from vinyl groups which are copolymerizable with at least one A monomer and at least one B monomer,

Y is chosen from divalent allylene groups, divalent arylene groups, divalent alkarylene groups, and divalent aralkylene groups, wherein the groups comprise from 1 to 30 carbon atoms, and further wherein the groups optionally further comprise at least one group chosen from ester groups, amide groups, urethane groups, and urea groups;

n is zero or 1;

m is a number ranging from 1 to 3;

R, which may be identical or different, are each chosen from hydrogen, C₁-C₄ alkyl groups, aryl groups, and alkoxy groups; and

Z, which may be identical or different, are each chosen from monovalent siloxane polymeric groups; and

D, which may be identical or different, are each chosen from free-radically-polymerizable acrylate copolymers and free-radically-polymerizable methacrylate copolymers. Such polymers and their manufacture are disclosed in U.S. Pat. Nos. 5,209,924 and 4,972,037, and WO 01/32737, the disclosures of which are hereby incorporated by reference.

Further non-limiting examples include polymers comprising at least one A monomer, at least one C monomer, and at least one D monomer, wherein A, which may be identical or different, are each chosen from polymerizable acrylic esters of at least one fluoroalkylsulfonamido alcohol and polymerizable methacrylic esters of at least one fluoroalkylsulfonamido alcohol, D, which may be identical or different, are each chosen from methacrylic acid esters of at least one C₁-C₁₂ linear alcohol and methacrylic acid esters of at least one C₁-C₂ branched alcohol, and C is as defined above in paragraphs 115 to 123. Such polymers include polymers comprising at least one group of the formula:

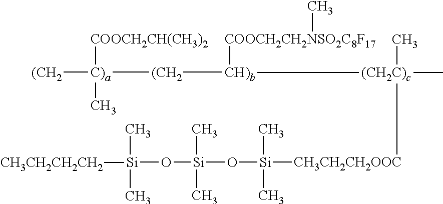

wherein

a, b, and c, which may be identical or different, are each a number ranging from 1 to 100,000; and

the terminal groups, which may be identical or different, are each chosen from C₁-C₂₀ linear alkyl groups, C₃-C₂₀ branched chain alkyl groups, C₃-C₂₀ aryl groups, C₁-C₂₀ linear alkoxy groups, and C₃-C₂₀ branched alkoxy groups.

Such polymers are disclosed in U.S. Pat. Nos. 4,972,037, 5,061,481, 5,209,924, 5,849,275, and 6,033,650, and WO 93/23446 and WO 95/06078, the disclosures of which are hereby incorporated by reference. These polymers may be purchased from Minnesota Mining and Manufacturing Company under the tradenames “Silicone Plus” polymers. For example, poly(isobutyl methacrylate-co-methyl FOSEA)-g-poly(dimethylsiloxane) is sold under the tradename SA 70-5 IBMMF.

Other non-limiting examples include silicone/acrylate graft terpolymers, for example, those having the formula:

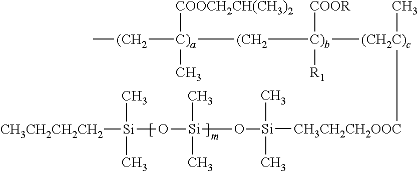

wherein

a, b, and c are present in a weight ratio of 69.9:0.1:30 respectively,

R and R¹, which may be identical or different, are each chosen from hydrogen and C₁-C₆ alkyl groups; and

m is a number ranging from 100-150.

According to preferred embodiments, m is chosen to provide a macromer having a molecular weight ranging from 8,000 to 12,000, such as 10,000. In another embodiment, m is a number ranging from 124-135, such as 130. Non-limiting examples of these copolymers are described in WO 01/32727 A1, the disclosure of which is hereby incorporated by reference.

According to other preferred embodiments, the polymer comprises a backbone chosen from vinyl backbones, methacrylic backbones, and acrylic polymeric backbones and further comprises at least one pendant siloxane group. Non-limiting examples of such polymers are disclosed in U.S. Pat. Nos. 4,693,935, 4,981,903, 4,981,902, the disclosures of which are hereby incorporated by reference.

In preferred embodiments the polymer comprises at least one A monomer, at least one C monomer, and, optionally at least one B monomer, wherein the at least one A monomer is chosen from free-radically-polymerizable vinyl monomers, free-radically-polymerizable methacrylate monomers, and free-radically-polymerizable acrylate monomers; the at least one B monomer, if present, is chosen from at least one reinforcing monomer copolymerizable with the at least one A monomer, and the at least one C monomer is chosen from monomers having the formula:

X(Y)ₙSi(R)₃₋ₘZₘ

wherein:

X is chosen from vinyl groups which are copolymerizable with the at least one A monomer and with the at least one B monomer;

Y is chosen from divalent groups;

n is zero or 1;

m is a number ranging from 1 to 3;

R, which may be identical or different, are each chosen from hydrogen, optionally substituted C₁-C₁₀ alkyl groups, optionally substituted phenyl groups, and optionally substituted C₁-C₁₀ alkoxy groups; and

Z, which may be identical or different, are each chosen from monovalent siloxane polymeric groups.

Non-limiting examples of A monomers include methacrylic acid esters of C₁-C₁₂ linear alcohols, methacrylic acid esters of C₁-C₁₂ of branched alcohols, styrene monomers, vinyl esters, vinyl chloride monomers, vinylidene chloride monomers, and acryloyl monomers.

Non-limiting examples of B monomers include acrylic monomers comprising at least one group chosen from hydroxyl, amino, and ionic groups, and methacrylic monomers comprising at least one group chosen from hydroxyl, amino, and ionic groups. Non-limiting examples of ionic groups include quaternary ammonium groups, carboxylate salts, and sulfonic acid salts.

The C monomers are as above defined above.

In yet another preferred embodiment, the polymer is chosen from vinyl-silicone graft copolymers having the following formula and vinyl-silicone block copolymers having the following formula:

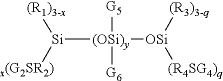

wherein

G₅, which may be identical or different, are each chosen from alkyl groups, aryl groups, aralkyl groups, alkoxy groups, alkylamino groups, fluoroalkyl groups, hydrogen, and —ZSA groups, wherein,

A is chosen from vinyl polymeric segments comprising at least one polymerized free-radically-polymerizable monomer, and

Z is chosen from divalent C₁-C₁₀ alkylene groups, divalent aralkylene groups, divalent arylene groups, and divalent alkoxylalkylene groups. In a preferred embodiment Z is chosen from methylene groups and propylene groups.

G₆, which may be identical or different, are each chosen from alkyl groups, aryl groups, aralkyl groups, alkoxy groups, alkylamino groups, fluoroalkyl groups, hydrogen, and —ZSA groups, as defined above;

G₂ comprises A;

G₄ comprises A.

R₁, which may be identical or different, are each chosen from alkyl groups, aryl groups, aralkyl groups, alkoxy groups, alkylamino groups, fluoroalkyl groups, hydrogen, and hydroxyl. In one embodiment, R₁ is chosen from C₁-C₄ alkyl groups, such as methyl groups, and hydroxyl.

R₂, which may be identical or different, are each chosen from divalent C₁-C₁₀ alkylene groups, divalent arylene groups, divalent aralkylene groups, and divalent alkoxyalkylene groups. In one embodiment, R₂ is chosen from divalent C₁-C₃ alkylene groups and divalent C₇-C₁₀ aralkylene groups. In another embodiment, R₂ is chosen from —CH₂— groups and divalent 1,3-propylene groups.

R₃, which may be identical or different, are each chosen from alkyl groups, aryl groups, aralkyl groups alkoxy groups, alkylamino groups, fluoroalkyl groups, hydrogen, and hydroxyl. In one embodiment, R₃ is chosen from C₁-C₄ alkyl groups and hydroxyl. In another embodiment, R₃ is chosen from methyl groups.

R4, which may be identical or different, are each chosen from divalent C₁-C₁₀ alkylene groups, divalent arylene groups, divalent aralkylene groups, and divalent alkoxyalkylene groups. In one embodiment, R₄ is chosen from divalent C₁-C₃ alkylene groups and divalent C₇-C₁₀ aralkylene groups. In another embodiment, R₄ is chosen from divalent —CH₂— groups and divalent 1,3-propylene groups.

x is a number ranging from 0 to 3;

y is a number greater than or equal to 5. In an embodiment, y ranges from 10 to 270, and in another embodiment, y ranges from 40 to 270.

q is a number ranging from 0 to 3;

Non-limiting examples of these polymers are described in U.S. Pat. No. 5,468,477, the disclosure of which is hereby incorporated by reference. A non-limiting example of such polymers is poly(dimethylsiloxane)-g-poly(isobutyl methacrylate), which is commercially available from 3M Company under the tradename VS 70 IBM.

The silicone acrylate polymers may be present in an amount ranging from 1 to 80% by weight, preferably from 5 to 70% by weight, more preferably from 10 to 65% by weight, more preferably from 10 to 60% by weight, and more preferably from to 50% by weight, based on the weight of the total composition.

The Solvent

The at least one solvent for use in the present invention may be chosen from volatile solvents and non-volatile solvents.

Volatile Solvents

The expression “volatile solvent” means any nonaqueous medium capable of evaporating from the keratinous material, at room temperature. This volatile phase in particular comprises oils with a vapor pressure at room temperature and at atmospheric pressure ranging from 0.13 Pa to 40,000 Pa (10⁻³ to 300 mm Hg). These volatile oils especially facilitate the application of the composition to the skin. These oils may be hydrocarbon-based oils, silicone oils (optionally comprising alkyl or alkoxy groups that are pendant or at the end of a silicone chain), and fluoro oils.

As volatile silicone oils, mention may be made of linear or cyclic silicones containing from 2 to 7 silicon atoms, these silicones optionally comprising alkyl or alkoxy groups containing from 1 to 10 carbon atoms. Mention may thus be made especially of octamethylcyclotetrasiloxane, decamethylcyclopentasiloxane, hexadecamethylcyclohexasiloxane, heptamethylhexyltrisiloxane and heptamethyloctyltrisiloxane, and mixtures thereof.

Volatile hydrocarbon-based oils that may be mentioned include C₈-C₁₆ isoparaffins such as the Isopar and Permethyl products, and especially isododecane, isooctane, isodecane and isohexadecane, and mixtures thereof.

Non-volatile Solvent

Suitable non-volatile solvents include, but are not limited to, various types of oils. Mention may be made of hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyldodecanol; silicone oils such as polydimethylsiloxanes (PDMS), which are optionally phenylated such as phenyltrimethicones, trimethyl pentaphenyl siloxane, tetramethyl tetraphenyl siloxane, or optionally substituted with aliphatic and/or aromatic groups that are optionally fluorinated, or with functional groups such as hydroxyl, thiol and/or amine groups; polysiloxanes modified with fatty acids, with fatty alcohols, with polyoxyalkylenes or with hydrocarbyl functional groups, fluorosilicones and perfluoro oils.

The at least one solvent will typically be present in the composition of the invention in an amount of from 10 to 90% by weight, preferably from 20 to 80% by weight, more preferably from 30 to 70% by weight, and more preferably from 40 to 70% by weight, based on the weight of the composition.

Colorants

The composition according to the invention may also comprise at least one colorant. The colorant may be chosen from the lipophilic dyes, hydrophilic dyes, pigments and nacres usually used in cosmetic or dermatological compositions, and mixtures thereof. The colorant is generally present in an amount of from 0.1 to 50% by weight, preferably from 0.5 to 30% by weight, and more preferably from 1 to 20% by weight, based on the weight of the composition.

The liposoluble dyes may be selected, for example, from Sudan Red, D&C Red 17, D&C Green 6, β-carotene, Sudan brown, D&C Yellow 11, D&C Violet 2, D&C Orange 5, quinoline yellow and annatto, or mixtures thereof.

The pigments may be white or colored, mineral and/or organic, coated or uncoated, and of usual or nanometric size. The term “pigments” should be understood as meaning particles that are insoluble in the medium, intended to color and/or opacify the composition. Among the mineral pigments that may be mentioned are titanium dioxide, optionally surface-treated, zirconium oxide or cerium oxide and also iron oxide or chromium oxide, manganese violet, ultramarine blue, chromium hydrate and ferric blue, and mixtures thereof. Among the organic pigments that may be mentioned are carbon black, pigments of D&C type, and lakes based on cochineal carmine or on barium, strontium, calcium or aluminum, and mixtures thereof. The pigments may especially be coated with at least one silicone compound such as polydimethylsiloxanes and/or with polymers, especially polyethylenes and/or at least one fluoro compound and/or at least one amino acid. Mention may also be made of “SI oxides” which are polymethylhydrogenosiloxane-coated pigments sold by the company Miyoshi.

The nacreous pigments, sometimes referred to as pearls in the cosmetic art, may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride, and mixtures thereof.

Other pigments which may be used include interference pigments and goniochromatic pigments such as those disclosed in US 20030064039 and US 20040241118, the entire contents of which are incorporated by reference.

The film forming polypropylsilsesquioxane resin used in the compositions of the invention is incorporated into cosmetically acceptable compositions which may be anhydrous, or in the emulsion form. If the latter, the emulsion may be water-in-oil (W/O) or oil-in-water (O/W) or a multiple emulsion, such as, for example O/W/O or W/O/W. Such emulsions will typically contain about 0.1-99% by weight water and 0.1-99% by weight oil, all weights being based on the weight of the composition.

Cosmetically Acceptable Additives

In addition to the above-mentioned ingredients, the cosmetic composition of the present invention may additionally contain various cosmetically acceptable additives.

Pasty Fatty Substance

The composition according to the invention may also contain at least one fatty compound that is pasty at room temperature. For the purposes of the invention, the expression “pasty fatty substance” means a compound with a melting point ranging from 25 to 60° C. and preferably from 30 to 45° C. and/or a hardness ranging from 0.001 to 0.5 MPa and preferably from 0.005 to 0.4 MPa.

The melting point values correspond to the melting point measured using a differential scanning calorimeter (DSC), such as the calorimeter sold under the name DSC 2920 by the company TA Instruments, with a temperature rise of 5 or 10° C. per minute. (The melting point considered is the point corresponding to the temperature of the most endothermic peak in the thermogram).

The hardness is measured according to a method of penetration of a probe into a sample of compound and in particular using a texture analyzer (for example the TA-XT2i from Rheo) equipped with a stainless steel cylinder 2 mm in diameter. The hardness measurement is performed at 20° C. at the center of 5 samples. The cylinder is introduced into each sample at a pre-speed of 1 mm/sec and then at a measuring speed of 0.1 mm/sec, the depth of penetration being 0.3 mm. The hardness value recorded is that of the maximum peak of the applied force.

According to the invention, one or more pasty fatty substances may also be used. Preferably, these fatty substances are hydrocarbon-based compounds, optionally of polymeric type; they may also be chosen from hydrocarbon-based compounds, silicone compounds and/or fluoro compounds, and mixtures thereof.

Among the pasty compounds that may be mentioned are lanolins and lanolin derivatives, for instance acetylated lanolins or oxypropylenated lanolins, with a viscosity from 18 to 21 Pa·s and preferably 19 to 20.5 Pa·s, and/or a melting point from 30 to 45° C., and mixtures thereof. Esters of fatty acids or of fatty alcohols may also be used, especially those containing 20 to 65 carbon atoms (melting point of about 20 to 35° C. and/or viscosity at 40° C. ranging from 0.1 to 40 Pa·s), for instance triisostearyl or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixin R” from Rheox.

Mention may also be made of silicone pasty fatty substances such as polydimethylsiloxanes (PDMS) containing pendant chains of the alkyl or alkoxy type containing from 8 to 24 carbon atoms, and having a melting point of 20-55° C., for instance stearyl dimethicones, especially those sold by the company Dow Corning under the trade names DC2503 and DC2-5514, and mixtures thereof.

The pasty fatty substance may be present in an amount of from 0.1 to 70% by weight, preferably from 1 to 50% by weight, and more preferably from 2 to 30% by weight, based on the weight of the composition.

Additional Film Formers

The composition according to the invention may also contain at least one additional film-forming polymer chosen from liposoluble film-forming polymers, lipodispersible film-forming polymers and mixtures thereof.

Liposoluble Polymer

The liposoluble polymers may be of any chemical nature and especially include:

1) liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄-C₅₀ alkyl group. The liposoluble homopolymers and copolymers may be chosen from those obtained from monomers chosen from the group consisting of isooctyl (meth)acrylate, isononyl (meth)acrylate, 2-ethylhexyl (meth)acrylate, lauryl (meth)acrylate, isopentyl (meth)-acrylate, n-butyl (meth)acrylate, isobutyl (meth)acrylate, tert-butyl (meth)acrylate, tridecyl (meth)acrylate and stearyl (meth)acrylate, or mixtures thereof. Examples that will be mentioned include the alkyl acrylate/cycloalkyl acrylate copolymer sold by PHOENIX CHEM under the name GIOVAREZ AC-5099 ML.

Liposoluble film-forming polymers that may also be mentioned include vinylpyrrolidone (VP) copolymers and especially copolymers of vinylpyrrolidone and of a C₂-C₄₀ and in particular C₃ to C₂₀ alkene. As examples of VP copolymers that may be used in the invention, mention may be made of copolymers of VP/vinyl acetate, VP/ethyl methacrylate, VP/ethyl methacrylate/methacrylic acid, VP/eicosene, VP/hexadecene, VP/triacontene, VP/styrene, VP/acrylic acid/lauryl methacrylate and butylated polyvinylpyrrolidone (PVP).

Particular liposoluble copolymers that may also be mentioned include:

i) liposoluble polymers belonging to one of the classes described above and bearing fluoro groups, in particular those described in U.S. Pat. No. 5,948,393 and the alkyl (meth)acrylate/perfluoroalkyl (meth)acrylate copolymers described in patents EP815836 and U.S. Pat. No. 5,849,318, the entire contents of which are incorporated by reference,

ii) polymers or copolymers resulting from the polymerization or copolymerization of an ethylenic monomer, comprising one or more ethylenic bonds, which are preferably conjugated (or diene). This or these agent(s) is (are) in particular vinyl, acrylic or methacrylic copolymers, which may be in block form and especially of diblock or triblock type, or even of multiblock or starburst type.

The ethylenic film-forming polymer may especially comprise a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

In particular, the film-forming polymer used may be a copolymer comprising at least one styrene block. Most particularly, a triblock copolymer may be used and in particular those of the polystyrene/polyisoprene or polystyrene/polybutadiene type, such as those sold under the name “LUVITOL HSB” by BASF, and those of the polystyrene/copoly(ethylene-propylene) type or alternatively of the polystyrene/copoly(ethylene-butylene) type, such as those sold under the brand name “KRATON” by Shell Chemical Co. or Gelled Permethyl 99A by Penreco. Styrene-methacrylate copolymers may also be used.

As film-forming polymers that may be used in the composition of the invention, examples that may also be mentioned include KRATON G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SiS), Gelled Permethyl 99A-750, Gelled Permethyl 99A-753-58 (blend of triblock and of starburst block polymer), Gelled Permethyl 99A-753-59 (blend of triblock and of starburst block polymer), Versagel 5970 and Versagel 5960 from Penreco (blend of triblock and of starburst polymer in isododecane), and OS 129880, OS 129881 and OS 84383 from Lubrizol (styrene-methacrylate copolymer);

2) amorphous liposoluble polysaccharides comprising alkyl (ether or ester) side chains, in particular alkylcelluloses comprising a linear or branched, saturated or unsaturated C₁-C₈ alkyl radical such as ethylcellulose and propylcellulose.

In general, the film-forming liposoluble polymers of the invention may have a weight-average molecular weight ranging from 1,000 to 500,000 and especially from 2,000 to 250,000, and a glass transition temperature ranging from −100° C. to +300° C., especially from −50° C. to +100° C. and in particular from −10° C. to +90° C.

Lipodispersible Polymer

The lipodispersible polymer is generally present in the form of a stable dispersion of particles, which are generally spherical and solid, in the liquid fatty phase. These dispersions may especially be in the form of polymer nanoparticles in dispersion. These nanoparticles may have a size ranging from 5 to 600 nm and especially ranging from 50 to 250 nm.

The dispersed polymer particles that may be used in the composition according to the invention may have a weight-average molecular weight ranging from about 2,000 to 10,000,000.

The polymer may have a glass transition temperature ranging from −100° C. to +300° C., especially from −10° C. to +50° C. and more particularly less than or equal to about +40° C.

The polymer used in the present invention in the form of particles dispersed in the fatty phase may be of any nature. Thus, a free-radical polymer, a polycondensate, or even a polymer of natural origin, and blends thereof, may be used. The polymer may be chosen by a person skilled in the art on the basis of its properties.

It is more particularly a “film-forming” polymer, i.e. a polymer capable of forming, alone or in combination with a plasticizer, an isolable film.

Illustrations of film-forming polymers that may be mentioned include acrylic or vinyl free-radical homopolymers and copolymers, especially those with a glass transition temperature Tᵤ of less than or equal to about +40° C. and in particular ranging from −10° C. to +30° C., and blends thereof.

The expression “free-radical polymer” means a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

Monomers bearing an acidic group that may be used include α, β-ethylenic unsaturated carboxylic acids such as acrylic acid, methacrylic acid, crotonic acid, maleic acid and itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), for instance (meth)acrylates of an alkyl, in particular of a C₁-C₂₀ and preferably C₁-C₈ alkyl, (meth)acrylates of an aryl, in particular of a C₆-C₁₀ aryl, (meth)acrylates of a hydroxyalkyl, in particular of a C₂-C₆ hydroxyalkyl.

Alkyl (meth)acrylates that may be mentioned include methyl, ethyl, butyl, isobutyl, 2-ethylhexyl and lauryl (meth)acrylate.

Hydroxyalkyl (meth)acrylates that may be mentioned include hydroxyethyl (meth)acrylate and 2-hydroxypropyl (meth)acrylate.

Aryl (meth)acrylates that may be mentioned include benzyl or phenyl acrylate.

Particularly advantageous (meth)acrylic acid esters are alkyl (meth)acrylates.

Free-radical polymers that may especially be used include copolymers of (meth)acrylic acid and of an alkyl (meth)acrylate, especially of a C₁-C₄ alkyl. More particularly, methyl acrylates optionally copolymerized with acrylic acid, such as copolymers of poly(methyl acrylate/acrylic acid) type, may be used.

Amides of the acidic monomers that may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide, and N-di(C₁-C₄)alkyl-(meth)acrylamides.

The vinyl polymers may also result from the polymerization of ethylenically unsaturated monomers containing at least one amine group, in free form or in partially or totally neutralized form, or alternatively in partially or totally quaternized form. Such monomers may be, for example, chosen from dimethylaminoethyl (meth)acrylate, dimethylaminoethylmethacrylamide, vinylamine, vinylpyridine or diallyldimethylammonium chloride.

The vinyl polymers may also result in the homopolymerization or copolymerization of at least one monomer chosen from vinyl esters and styrene monomers. In particular, these monomers may be polymerized with acidic monomers and/or esters thereof and/or amides thereof, such as those mentioned above.

Examples of vinyl esters that may be mentioned include vinyl acetate, vinyl propionate, vinyl neodecanoate, vinyl pivalate, vinyl benzoate and vinyl t-butylbenzoate.

Styrene monomers that may be mentioned include styrene and α-methylstyrene.

The list of monomers given is not limiting and it is possible to use any monomer known to those skilled in the art falling within the categories of acrylic and vinyl monomers (including monomers modified with a silicone chain).

As other vinyl monomers that may be used, mention may also be made of:

N-vinylpyrrolidone, vinylcaprolactam, vinyl-N—(C₁-C₆) alkylpyrroles, vinyloxazoles, vinylthiazoles, vinylpyrimidines and vinylimidazoles, and

olefins such as ethylene, propylene, butylene, isoprene and butadiene.

The vinyl polymer may be crosslinked with the aid of difunctional monomers especially comprising at least two ethylenic unsaturations, such as ethylene glycol dimethacrylate or diallyl phthalate.

In a nonlimiting manner, the polymers of the invention may be chosen from the following polymers or copolymers: polyurethanes, polyurethane-acrylics, polyureas, polyurea-polyurethanes, polyester-polyurethanes, polyether-polyurethanes polyesters, polyesteramides, alkyd fatty-chain polyesters; polyacrylamides; silicone polymers, fluoro polymers, and mixtures thereof.

According to one particular variant of the invention, the lipodispersible polymers may be surface-stabilized with at least one liposoluble polymer, for instance those described below or those described in patent application EP 1002528, the entire contents of which are incorporated by reference.

The film-forming polymer dispersion may be prepared as described in document EP749747 or in document EP1002528. More specifically, the polymerization is performed in dispersion, i.e. by precipitation of the polymer as it is formed, with protection of the formed particles with a stabilizer. Stabilizers are especially described in EP749747, the entire contents of which are incorporated by reference.

In general, the composition according to the invention may comprise from 0.1 to 30% by weight, preferably from 0.5 to 20% by weight, and more preferably from 1 to 15% by weight (expressed as solids), based on the weight of the composition, of a film-forming polymer and especially a lipodispersible film-forming polymer.

Structuring Agents

The composition of the invention may also contain structuring agents. Structuring agents are compounds which help with the integrity and the strength of the composition. Structuring agents may include waxes and polyamides.

i) Waxes

For the purposes of the present invention, a wax is a lipophilic fatty compound, which is solid at room temperature (25° C.) with a reversible solid/liquid change of state, having a melting point of greater than 45° C. and better still greater than 55° C., which may be up to 200° C., and having an anisotropic crystal organization in the solid state. For the purposes of this invention, the waxes are those generally used in cosmetics and dermatology. The waxes may be of natural origin, for instance beeswax, carnauba wax, candelilla wax, ouricoury wax, Japan wax, cork fiber wax or sugar cane wax, paraffin wax, lignite wax, microcrystalline waxes, lanolin wax, montan wax, ozokerites and hydrogenated oils, for instance hydrogenated jojoba oil. They may also be of synthetic origin, for instance polyethylene waxes derived from the polymerization of ethylene, waxes obtained by Fischer-Tropsch synthesis, esters of fatty acids and of glycerides that are solid at 40° C.

They may also be silicone waxes, referred to as alkyl, alkoxy or esters of poly(di)methylsiloxane silicones, which are polymers that comprise repeating dimethylsiloxy units in combination alkyl siloxy units wherein the long chain alkyl is generally a fatty chain that provides a wax-like character to the silicone. Such silicones include, but are not limited to stearoxydimethicone, behenoxy dimethicone, stearyl dimethicone, cetearyl dimethicone, and the like which are solid at 40° C. Waxes of synthetic origin are preferably used for reasons of greater reproducibility than waxes of natural origin.

ii) Polyamides

Polyamides are compounds which result from the condensation, via an amide bond of carbonyl and amine groups. Substitution may be possible on the molecule, i.e. as a pendant chain on the molecule or on the terminal ends, or within the molecule, making it a copolymer.

Polyamide polymers resulting from the condensation of at least one aliphatic dicarboxylic acid and at least one diamine may be used Examples of these polyamide polymers are those sold under the brand name Versamid by the companies General Mills Inc. and Henkel Corp. (Versamid 930, 744 or 1655) or by the company Olin Mathieson Chemical Corp. under the brand name Omamid, in particular Omamid S or C. These resins have a weight-average molecular mass ranging from 6000 to 9000. For further information regarding these polyamides, reference may be made to U.S. Pat. Nos. 3,645,705 and 3,148,125, the entire contents of which are incorporated by reference.

Other examples of polyamides include those sold by the company Arizona Chemicals under the references Uni-Rez (2658, 2931, 2970, 2621, 2613, 2624, 2665, 1554, 2623 and 2662) and the product sold under the reference Macromelt 6212 by the company Henkel. These polyamides are described in U.S. Pat. No. 5,500,209, the entire contents of which are incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins disclosed in U.S. Pat. Nos. 5,783,657 and 5,998,570, the entire contents of which are incorporated by reference.

Other structuring agents which may be used alone or in combination with the above are non silicone polyamides such as those known in the trade as Uniclear or Sylvaclear. These non silicone polyamides have different terminal end groups, such as ester terminated, known as Uniclear 80 or 100, such as amide terminated, known as Sylvaclear A200, and such as polyalkyleneoxy terminated, known as Sylvaclear AF1900 as well as ester terminated polyesteramides. These non silicone polyamides are available, for instance, from Arizona Chemical Company, Jacksonville, Fla., and are described in U.S. Pat. Nos. 5,783,657, 6,402,408, 6,268,466 and 6,552,160, the entire contents of which are incorporated by reference.

In the event that a structuring agent is employed, it may be present in the composition in an amount of from 0.1 to 30% by weight, preferably from 1 to 20% by weight, and more preferably from 2 to 15% by weight, based on the weight of the composition.

Organogelator

The composition of the invention may also comprise at least one organogelator. An organogelator is defined herein to include a non-polymeric organic compound whose molecules may be capable of establishing, between themselves, at least one physical interaction leading to a self-aggregation of the molecules with formation of a macromolecular 3-D network which may be responsible for the gelation of the liquid fatty phase. The network can result from the formation of a network of fibrils (due to the stacking or aggregation of organic-gelling molecules), immobilizing the molecules of the liquid fatty phase. Depending on the nature of the organogelator, the interconnected fibrils have variable dimensions which may range up to one micron, or even several microns. These fibrils may occasionally combine to form strips or columns.

The term “gelation” means a thickening of the medium which may result in a gelatinous consistency and even in a solid, rigid consistency which does not flow under its own weight. The ability to form this network of fibrils, and thus the gelation, depends on the nature (or chemical category) of the organogelator, the nature of the substituents borne by its molecules for a given chemical category, and the nature of the liquid fatty phase. For example, this gelation is reversible.

The physical interactions are diverse but may exclude co-crystallization. These physical interactions are, for instance, interactions chosen from self-complementary hydrogen interactions, π interactions between unsaturated rings, dipolar interactions, and coordination bonding with organometallic derivatives. The establishment of these interactions may often be promoted by the architecture of the molecule, such as by rings, unsaturations, and the presence of asymmetric carbons. In general, each molecule of an organogelator can establish several types of physical interaction with a neighboring molecule. Thus, in one embodiment, the molecules of the organogelator according to the invention may comprise at least one group capable of establishing hydrogen bonding, e.g., at least two groups capable of forming hydrogen bonding; at least one aromatic ring, e.g., at least two aromatic rings; at least one bond with ethylenic unsaturation; and/or at least one asymmetric carbon. The groups capable of forming hydrogen bonding may, for example, be chosen from hydroxyl, carbonyl, amine, carboxylic acid, amide and benzyl groups.

The organogelator of the invention may be soluble in the liquid fatty phase of the composition at room temperature and atmospheric pressure. It may be solid or liquid at room temperature and atmospheric pressure.

Organogelator(s) which can be used in the invention are, for example, those described in the document “Specialist Surfactants” edited by D. Robb, 1997, pp. 209-263, chapter 8, by P. Terech, and U.S. Pat. Nos. 6,372,235 and 6,726,195, the entire contents of which are incorporated by reference.

In the event that an organogelator is employed, it may be present in the composition in an amount of from 0.1 to 20_% by weight, preferably from 0.2 to 10% by weight, and more preferably from 0.5 to 5% by weight, based on the weight of the composition.

Gelling Agents

The composition of the invention may also contain at least one agent useful for gelling a liquid fatty phase. The gelling agent increases the liquid fatty phase viscosity and leads to a solid or flowable composition when introduced in said fatty phase. The gelling agent does not encompass waxes, in the sense that it is not waxy.

The at least one gelling agent may be chosen from gelling agents in polymeric form and gelling agents in mineral form.

In one embodiment, the at least one gelling agent is not soluble in an aqueous phase or in water.

The gelling agent according to the present invention is preferably selected from the group consisting of agents that gel via chemical reticulation and agents that gel via physical reticulation.

Gelling Agents that Gel Via Chemical Reticulation

According to one embodiment, crosslinked elastomeric polyorganosiloxanes of three-dimensional structure are preferred. These elastomeric silicones can bear hydrophilic groups, such as polyoxyethylene or copoly(oxyethylene/oxypropylene).

As elastomeric polyorganosiloxanes which can be used in the invention, mention may be made of the crosslinked elastomeric polyorganosiloxanes described in application EP295886A, the entire contents of which are incorporated by reference. According to that application, they are obtained by addition reaction and crosslinking, in the presence of a platinum-type catalyst, of at least:
(a) a polyorganosiloxane having at least two C₂ to C₆ lower alkenyl groups per molecule; and
(b) a polyorganosiloxane having at least two hydrogen atoms linked to a silicon atom per molecule.

It is also possible to use the polyorganosiloxanes described in U.S. Pat. No. 5,266,321, the entire contents of which are incorporated by reference. According to that patent, they are chosen in particular from:
i) polyorganosiloxanes comprising R₂SiO and RSiO_{1.5} units and optionally R₃SiO_{0.5} and/or SiO₂ units in which the radicals R, independently of each other, are chosen from a hydrogen, an alkyl such as methyl, ethyl or propyl, an aryl such as phenyl or tolyl, an unsaturated aliphatic group such as vinyl, the weight ratio of the units R₂SiO to the units RSiO_{1.5} ranging from 1/1 to 30/1;
ii) polyorganosiloxanes which are insoluble and swellable in silicone oil, obtained by addition of an polyorganohydrogenosiloxane (1) and of a polyorganosiloxane (2) having unsaturated aliphatic groups such that the amount of hydrogen or of unsaturated aliphatic groups in (1) and (2) respectively ranges from 1 to 20 mol % when the polyorganosiloxane is non-cyclic and from 1 to 50 mol % when the polyorganosiloxane is cyclic. Optionally, these polyorganosiloxanes can comprise from 1 to 40 oxyalkylene groups, such as oxypropylene and/or oxyethylene groups.

As examples of elastomeric polyorganosiloxanes which can be used according to the invention, mention may be made of those sold or made under the names KSG6 from Shin-Etsu, Trefil E-505C or Trefil E-506C from Dow-Corning, Gransil from Grant Industries (SR-CYC, SR DMF10, SR-DC556) or those marketed in the form of preconstituted gels (KSG15, KSG17, KSG16, KSG18, KSG21 from Shin-Etsu, Gransil SR 5CYC gel, Gransil SR DMF 10 gel, Gransil SR DC556 gel, SF 1204 and JK 113 from General Electric or emulsifying elastomers such as those sold under the names of KSG-210, KSG-30, KSG-31, KSG-32, KSG-33, KSG-40, KSG 41, KSG-42, KSG-43 and KSG-44 from Shin-Etsu. A mixture of these commercial products may also be used.

Also available are cyclized dimethicones. The term “cyclized dimethicone” means an organosiloxane comprised of repeating —[SiO_{2/2}]—, or “D” units as defined previously, which form one or more cyclized portions in the final polymer. The cyclized portions, or rings, are formed by crosslinking certain portions along the organosiloxane chain to form rings that may be structurally aligned along the polymeric chain in the manner depicted below:

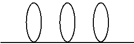

Preferably, the rings in the polymer have a molecular weight ranging from about 40,000 to 50,000, more preferably about 45,000, with the final polymer having a molecular weight ranging from about 1,600,000 to 2,600,000, preferably about 2,000,000. Cyclized dimethicone may be purchased from Jeen International under the tradename JEESILC IDD which is a mixture of cyclized dimethicone (having the INCI name dimethicone crosspolymer-3) and isododecane; or JEECHEM HPIB which is a mixture of cyclized dimethicone (dimethicone crosspolymer-3), hydrogenated polyisobutene, and cyclomethicone. The compositions of the invention may contain from about 0.1-95%, preferably about 0.5-80%, more preferably 1-75% by weight by the weight of total composition of the cyclized dimethicone. A mixture of these commercial products may also be used.

Gelling Agents that Gel Via Physical Reticulation

Gelling agents that gel via physical reticulation, in particular via molecular muddling, hydrogen interactions, sequences incompatibility or dipolar interactions, as well as liposoluble polymers having liquid crystal groups, are preferred.

Gelling agents that gel via molecular muddling are polymers having high molecular weights, preferably higher than 500,000, such as silicone gums.

The silicone gum can correspond to the formula:

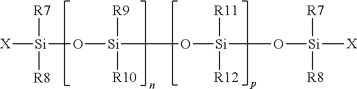

in which:

R₇, R₈, R₁₁ and R₁₂ are identical or different, and each is chosen from alkyl radicals comprising from 1 to 6 carbon atoms,

R₉ and R₁₀ are identical or different, and each is chosen from alkyl radicals comprising from 1 to 6 carbon atoms and aryl radicals,

X is chosen from alkyl radicals comprising from 1 to 6 carbon atoms, a hydroxyl radical and a vinyl radical,

n and p are chosen so as to give the silicone gum a viscosity of greater than 100,000 mPa·s, such as greater than 500,000 mPa·s.

In general, n and p can each take values ranging from 0 to 5,000, such as from 0 to 3,000.

Among the silicone gums which can be used according to the invention, mention may be made of those for which:

the substituents R₇ to R₁₂ and X represent a methyl group, p=0 and n=2,700, such as the product sold or made under the name SE30 by the company General Electric,

the substituents R₇ to R₁₂ and X represent a methyl group, p=0 and n=2,300, such as the product sold or made under the name AK 500000 by the company Wacker,

the substituents R₇ to R₁₂ represent a methyl group, the substituent X represents a hydroxyl group, p=0 and n=2,700, as a 13% solution in cyclopentasiloxane, such as the product sold or made under the name Q2-1401 by the company Dow Corning,

the substituents R₇ to R₁₂ represent a methyl group, the substituent X represents a hydroxyl group, p=0 and n=2,700, as a 13% solution in polydimethylsiloxane, such as the product sold or made under the name Q2-1403 by the company Dow Corning, and

the substituents R₇, R₈, R₁₁, R₁₂ and X represent a methyl group and the substituents R₉ and R₁₀ represent an aryl group, such that the molecular weight of the gum is about 600,000, for instance the product sold or made under the name 761 by the company Rhone-Poulenc (Rhodia Chimie).

In preferred embodiments, the silicone gum correspond to the following formula:

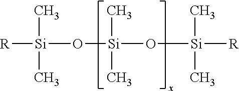

In this formula the terminal Si's can also be other than methyl and may be represented with substitutions on the repeating Si such that the R group is an alkyl of 1 to 6 carbon atoms, which may be linear, branched and/or functionalized selected from methyl, ethyl, propyl, isopropyl, butyl, isobutyl, t-butyl, amyl, hexyl, vinyl, allyl, cyclohexyl, phenyl, fluoroalkyl, and mixtures thereof. The silicone gums employed in the present invention may be terminated by triorganosilyl groups of the formula R′₃ where R′ is a radical of monovalent hydrocarbons containing from 1 to 6 carbon atoms, hydroxyl groups, alkoxyl groups and mixtures thereof. The silicone gums used in the invention have an affinity with the structuring polymer and/or with the silicone gum, and the liquid fatty phase, the polymer and the silicone gum form a physiologically acceptable medium.

A particularly preferred fluid diorganopolysiloxane polymer is poly(dimethylsiloxane), herein referred to as PDMS. Also useful is a mixture of silicone gums such as the commercially available DC 1503 which is a blend of dimethicone and dimethiconol. Other useful silicone gums are DC 1428 fluid (Dow Corning) and those silicone gums described in U.S. Pat. No. 4,574,082, the entire contents of which are incorporated by reference.

In certain embodiments of the present invention, crystalline silicone compounds are included in the compositions.

A crystalline silicone compound is a compound comprising silicone in its molecule, which is solid at room temperature, and has a crystalline character.

The crystalline silicone compounds belong to a class of alkyl siloxane waxes corresponding to the formulae below:

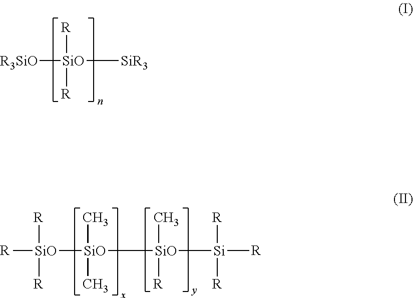

[(CH₃)₃SiO]₂(CH₃)SiR

(CH₃)₃SiO[(CH₃)₂SiO]ₓ(RCH₃SiO)_{y}Si(CH₃)₃

This could also be written as:

R₃SiO[(CH₃)₂SiO]ₓ(RCH₃SiO)_{y}SiR₃

where R is an alkyl chain. x may be 0. The substituent R may be as low as 1 or as high as 50 or more as long as this silicone compound crystallizes at room temperature.

Examples of crystalline silicone compounds include, but are not limited to, C20-24 Alkyl Methicone, C24-28 Alkyl Dimethicone, C20-24 Alkyl Dimethicone, C24-28 Alkyl Dimethicone commercially available from Archimica Fine Chemicals, Gainesville, Fla. under the designation of SilCare 41M40, SilCare 41M50, SilCare 41M70 and SilCare 41M80. Stearyl Dimethicone available as SilCare 41M65 from Archimica or as DC-2503 from Dow-Corning, Midland, Mich. Similarly, stearoxytrimethylsilane sold as SilCare 1M71 or DC-580 may be used in an embodiment of this invention. Furthermore, similar crystalline compounds are available from Degussa Care Specialties, Hopewell, Va. under the designation ABIL Wax 9810, 9800, or 2440, or Wacker-Chemie GmbH, Burghausen, Germany, under the designation BelSil SDM 5055, or OSi Specialties, Greenwich, Conn. under the designation Silsoft. Other crystalline silicone compounds include C30-45 Alkyl Methicone available from Dow Corning as AMS-C30 Wax, as well as GE's SF1642, or SF-1632 available from General Electric, Fairfield, Conn.

In another embodiment, the at least one gelling agent may be in mineral form with particle sizes that cause little or no light scattering. Thus, it may be possible to obtain a translucent or even transparent composition.

As modified clays which can be used, mention may be made of hectorites modified with an ammonium chloride of a C₁₀ to C₂₂ fatty acid, such as hectorite modified with distearyldimethylammonium chloride, also known as quaternium-bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as steralkonium bentonites, such as the products sold or made under the names Claytone APA and Claytone AF by the company Southern Clay, and Baragel 24 sold or made by the company Rheox.

As other mineral gelling agents, which can be used in the invention, mention may be made of silica, such as fumed silica. The fumed silica may have a particle size, which may be nanometric to micrometric, for example ranging from about 5 nm to 200 nm.

The fumed silicas may be obtained by high-temperature hydrolysis of a volatile silicon compound in a hydrogen-oxygen flame, producing a finely divided silica. This process makes it possible to obtain hydrophilic silicas that have a large number of silanol groups at their surface. Such hydrophilic silicas are sold or made, for example, under the names “Aerosil 130®”, “Aerosil 200®”, “Aerosil 255®”, “Aerosil 300®” and “Aerosil 380®” by the company Degussa, and “CAB-O-SIL HS-5®”, “CAB-O-SIL EH-5®”, “CAB-O-SIL LM-130®”, “CAB-O-SIL MS-55®” and “CAB-O-SIL M-5®” by the company Cabot.

It is thus possible to chemically modify the surface of the hydrophilic silica by chemical reaction, producing a reduction in the number of silanol groups. The silanol groups can be replaced, for example, with hydrophobic groups: this then gives a hydrophobic silica.

The hydrophobic groups may be:
trimethylsiloxyl groups, which are obtained in particular by treating fumed silica in the presence of hexamethyldisilazane. Silicas thus treated are known as “silica silylate” according to the CTFA. They are sold or made, for example, under the references “Aerosil R812®” by the company Degussa and “CAB-O-SIL TS-530®” by the company Cabot;
dimethylsilyloxyl or polydimethylsiloxane groups, which are obtained in particular by treating fumed silica in the presence of polydimethylsiloxane or dimethyldichlorosilane. Silicas thus treated are known as “silica dimethyl silylate” according to the CTFA (6th edition, 1995). They are sold or made, for example, under the references “Aerosil R972®” and “Aerosil R974®” by the company Degussa, and “CAB-O-SIL TS-610®” and “CAB-O-SIL TS-720®” by the company Cabot;
groups derived from reacting fumed silica with silane alkoxides or siloxanes. These treated silicas are, for example, the products sold or made under the reference “Aerosil R805®” by the company Degussa.

Hydrophobic silica, such as fumed silica, may be used as lipophilic gelling agent. The use of fumed silica makes it possible to obtain a translucent or even transparent composition, in particular in the form of a stick, which does not exude, in the absence of opacifying particles such as waxes, fillers and pigments (including nacres).

The at least one liposoluble gelling agent can allow the exudation of the composition to be limited and can allow its stability to be increased, while at the same time conserving the composition's glossy appearance, which is not possible with waxes such as those used conventionally in cosmetics and dermatology.

In the event that a gelling agent is employed, it may be present in the composition in an amount of from 0.1 to 40% by weight, preferably from 0.5 to 30% by weight, and more preferably from 1 to 20% by weight, based on the weight of the composition.

Emollients

Another useful component of cosmetic compositions is an emollient. These emollients may be silicone compatible oils that mimic or are compatible with a lipophilic content generally found within skin or hair. A preferred emollient is squalane. However, many traditional silicone compatible oils may be used. Useful emollients include, but are not limited to fatty bodies liquid at ambient temperature, such as esters, mineral oils, animal oils, vegetable oils, synthetic oils, and silicone oils. Examples of useful esters include, but are not limited to, isononyl isononanoate, octyl palmitate, cetyl lactate, pentaerythrityl tetraoctanoate, tridecyl octanoate, tridecyl behenate, isopropyl jojobate and jojoba alcohols, butyloctyl salicylate, polyglyceryl-3 diisostearate, tridecyl trimellitate, tridecyl stearate, and neopentylglycol dicaprylate/dicaprate. In one embodiment of the invention, the esters are chosen from isononyl isononanoate, a light ester which adds to the initial feel of the inventive composition on the skin, and pentaerythrityl tetraoctanoate, an ester which helps to cushion after the makeup is blended into the skin. Examples of useful oils include, but are not limited to, petrolatum oil, liquid lanolin, arara oil, sesame oil, macadamia oil, jojoba oil, silicone oils such as phenyl trimethicone and dimethicone, and synthetic triglycerides such as capric caprylic triglyceride and hydrogenated cocoglycerides.

The silicone compatible organic oil which may be used as emollients also include esters, hydrocarbon oils, or an animal, vegetable, or mineral oils. The term compatible means that the organic oil is soluble or dispersible in the silicone mixture to form a stable solution or dispersion. Esters include mono-, di-, and triesters. The composition may comprise one or more esters selected from the group, or mixtures thereof. Preferably the composition contains a mixture of di- and triesters. Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups, and in one preferred embodiment of the invention the acid is an α-hydroxy acid. Either one or both of the acid or alcohol may be a fatty acid or alcohol, i.e. may have from about 6 to 22 carbon atoms. Examples of monoester oils that may be used in the compositions of the invention include hexyl decyl benzoate, hexyl laurate, hexadecyl isostearate, hexyldecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid. Preferred is monoester which is the reaction product of an aliphatic C₂₋₈ alcohol and a C₁₄₋₂₂ fatty acid, more particularly, the reaction product of a hexyl alcohol and lauric acid, also referred to as hexyl laurate.

Suitable diesters that may be used in the compositions of the invention are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be substituted with one or more hydroxyl group. The aliphatic or aromatic alcohol may also contain 2 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms. The dicarboxylic acid may also be an α-hydroxy acid. Examples of diester oils that may be used in the compositions of the invention include diisostearyl malate (the reaction product of isostearic alcohol and malic acid), neopentyl glycol dioctanoate (the reaction product of neopentyl glycol and 2-ethyl hexanoic acid), dibutyl sebacate (reaction product of butyl alcohol and sebacic acid), di-C₁₂₋₁₃ alkyl malate (reaction product of C₁₂₋₁₃ alcohol and malic acid), dicetearyl dimer dilinoleate (reaction product of cetearyl alcohol and adipic acid), dicetyl adipate (reaction product of cetyl alcohol and adipic acid), diisocetyl adipate (reaction product of hexadecyl alcohol and adipic acid), diisononyl adipate (reaction product of isononyl alcohol and adipic acid), diisostearyl dimer dilinoleate (reaction product of isostearyl alcohol and dilinoleic acid), diisostearyl fumarate (reaction product of isostearyl alcohol and fumaric acid), and so on.

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 14 to 22 carbon atoms.

Examples of triesters include triarachidin (reaction product of glycerin and arachidic acid), tributyl citrate (reaction product of butyl alcohol and citric acid), tri C₁₂₋₁₃ alkyl citrate (reaction product of C₁₂₋₁₃ alcohol and citric acid), tricaprylin (reaction product of glycerin and caprylic acid), tricaprylyl citrate (reaction product of capryl alcohol and citric acid), tridecyl behenate (reaction product of tridecyl alcohol and behenic acid), trioctyldodecyl citrate (reaction product of octyldodecyl alcohol and citric acid), tridecyl behenate (reaction product of tridecyl alcohol and behenic acid), tridecyl cocoate (reaction product of tridecyl alcohol and coconut acid), tridecyl isononanoate (reaction product of tridecyl alcohol and isononanoate), and so on. Preferred is a triester which is the reaction product of an α-hydroxy acid and a guerbet alcohol having 6 to 30 carbon atoms, in particular the reaction product of citric acid and octyldodecyl alcohol, referred to as trioctyldodecyl citrate.

In the event that an emollient is employed, it may be present in the composition in an amount of from 0.1 to 80% by weight, preferably from 1 to 70% by weight, and more preferably from 2 to 60% by weight, based on the weight of the composition.

Hydrating Agents and Skin Conditioning Agents

Suitable hydrating agents and skin conditioning agents may include hydroxyl-containing compounds such as polyols, polymeric or monomeric ethers, dihydric alcohols, as well as cationic agents and the like.

Polyols

Polyols are suitable hydrating agents. For purposes of this specification, polyols are defined as compounds which contain three or more hydroxyl groups per molecule. Examples of suitable polyols include fructose, glucamine, glucose, glucose glutamate, glucuronic acid, glycerin, 1,2,6 hexanetriol, hydroxystearyl methylglucanine, inositol, lactose, maltitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and mixtures thereof. An especially preferred polyol is glycerin.

Dihydric Alcohols

Also suitable for use as hydrating agents are dihydric alcohols of the general formula R(OH)ₙ where n is 2 and R is a substituted or unsubstituted saturated C₂-C₁₀, preferably C₁-C₈ alkyl, or a substituted or unsubstituted alicyclic, bicyclic, or aromatic ring, with the substituents selected from halogen, alkoxy, hydroxy, and so on. Examples of suitable alcohols include hexylene glycol, diethylene glycol, ethylene glycol, propylene glycol, 1,2-butylene glycol, triethylene glycol, dipropylene glycol, and mixtures thereof.

Polymeric or Monomeric Ethers

Also suitable as hydrating agents are homopolymeric or block copolymeric liquid ethers. Polymeric ethers are preferably formed by polymerization of monomeric alkylene oxides, generally ethylene or propylene oxides. Examples of such polymeric ethers include PEG, PPG, and derivatives thereof.

Other examples of suitable polymeric ethers include polyoxypropylene polyoxyethylene block copolymers. Such compounds are sold under the CTFA name Meroxapol 105, 108, 171, 172, 174, 178, 251, 252, 254, 255, 258, 311, 312, and 314.

In the event that a hydroxyl-containing hydrating/skin conditioning agent is employed, it may be present in the composition in an amount of from 0.01 to 20% by weight, preferably from 0.5 to 15% by weight, and more preferably from 1 to 10% by weight, based on the weight of the composition.

Cationic Skin Conditioning Agents

Cationic skin conditioning agents may include cationic polymers such as cationic polysaccharides selected from the group consisting of celluloses, guars, their derivatives and their mixtures. Cationic cellulose is available from Amerchol Corp. (Edison, N.J., USA) in their Polymer JR™ and LR™ series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Another suitable type of cationic polysaccharide that can be used is a cationic guar gum derivative, such as guar hydroxypropyltrimonium chloride (Commercially available from Rhone-Poulenc in the JAGUAR™ series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

In the event that a cationic hydrating/skin conditioning agent is employed, it may be present in the composition in an amount of from 0.01 to 2% by weight, preferably from 0.1 to 1.5% by weight, and more preferably from 0.2 to 1% by weight, based on the weight of the composition.

Fillers

The composition may also contain at least one filler, especially in order to obtain a matt product, which is especially desired for foundations and in particular for foundations or day creams for individuals with greasy skin. The term “filler” means any particle that is solid at room temperature and atmospheric pressure, used alone or in combination, which does not react chemically with the various ingredients of the composition and which are insoluble in these ingredients, even when these ingredients are brought to a temperature above room temperature and especially to their softening point or to their melting point. These inert fillers have melting points at least higher than 170° C. and better still higher than 200° C. They may be absorbent or nonabsorbent, i.e. capable in particular of absorbing the oils of the composition and also the biological substances secreted by the skin. Preferably, these fillers have an apparent diameter ranging from 0.01 to 150 μm, preferably from 0.5 to 120 μm and better still ranging from 1 to 80 μm. An apparent diameter corresponds to the diameter of the circle in which the elementary particle is inscribed along its smallest dimension (thickness for lamellae).

The fillers that may be used in the composition according to the invention may be mineral or organic, lamellar, spherical or oblong. Mention may be made of talc, mica, silica, kaolin, polyamide powders, for instance Nylon®. (Orgasol® from Atochem), poly-β-alanine powders, polyethylene powders, powders of an acrylic polymer and especially of polymethyl methacrylate (PMMA), for instance the product sold by Wackherr under the reference Covabead LH-85 (particle size 10-12 μm), powders of acrylic acid copolymers (Polytrap® from Dow Corning), polytetrafluoroethylene (Teflon®) powders, lauroyllysine, boron nitride, starch, hollow polymer microspheres such as those of polyvinylidene chloride/acrylonitrile, for instance Expancel® (Nobel Industrie), carbonates such as precipitated calcium carbonate, magnesium carbonate and magnesium hydrocarbonate, hydroxyapatite, hollow silica microspheres (Silica Beads® from Maprecos), hollow silica ellipsoids (Silica Shells from Kobo), glass microcapsules, ceramic microcapsules and polyester particles, and mixtures thereof. These fillers may be surface-treated, especially to make them lipophilic.

In the event that a filler is employed, it may be present in the composition in an amount of from 0.1 to 50% by weight, preferably from 1 to 40% by weight, and more preferably from 2 to 30% by weight, based on the weight of the composition.

Surfactants

The composition according to the invention may contain a surfactant or a mixture of surfactants, especially a surfactant whose HLB (hydrophilic/lipophilic balance) value allows the production of a water-in-oil (W/O) or oil-in-water (O/W) emulsion.

As surfactants that may be used, suitable for obtaining a W/O emulsion, mention may be made of those with an HLB value of less than 7, and especially fatty acid esters of polyols, for instance mono-, di-, tri- or sesquioleates or stearates of sorbitol or of glycerol, glyceryl laurate or polyethylene glycol laurate; alkyl or alkoxy dimethicone copolyols with an alkyl or alkoxy chain pendant or at the end of the silicone skeleton, containing, for example, from 6 to 22 carbon atoms.

As surfactants that may be used to obtain an O/W emulsion, mention may be made of those with an HLB value of greater than 7, for instance fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers and dimethicone copolyols; and mixtures thereof. In general, any amphoteric ionic (cationic or anionic) surfactant and any nonionic surfactant that is well known to those skilled in the art may be used.

In the event that a surfactant is employed, it may be present in the composition in an amount of from 0.1 to 20% by weight, preferably from 0.5 to 15% by weight, and more preferably from 1 to 10% by weight, based on the weight of the composition.

The composition of the invention may also comprise any additive conventionally used in the field of cosmetics such as, for example, antioxidants, essential oils, preserving agents, fragrances, neutralizers, cosmetic or dermatological active agents such as, for example, vitamins, essential fatty acids, sunscreens, free-radical scavengers, dispersants, anti-acne agents and mixtures thereof. These additives may be present in the composition in an amount of from 0.01 to 10% by weight, preferably from 0.1% to 8% by weight, and more preferably from 0.5 to 5% by weight, based on the total weight of the composition. Advantageously, the composition contains at least one cosmetic or dermatological active agent.

Needless to say, a person skilled in the art will take care to select the optional additional additives and/or the amount thereof such that the advantageous properties of the composition according to the invention are not, or are not substantially, adversely affected by the envisaged addition.

### EXAMPLES

The present invention will be better understood from the examples which follow, all of which are intended for illustrative purposes only, and are not meant to unduly limit the scope of the invention in any way.

### Examples 1-3

### Lip Compositions

| | | | Ex. 1 | Ex. 2 | Ex. 3 |
|---|---|---|---|---|---|
| PHASE | Trade Name | INCI Name | w/w % | w/w % | w/w % |
| A | DOW | Isododecane (and) | 25.00 | 28.10 | 38.00 |
| | CORNING | Polypropyl- | | | |
| | DC 670 | silsesquioxane | | | |
| | | (80% solids) | | | |
| | DC2-8179 | Nylon-611/ | 5.00 | 2.50 | 1.50 |
| | | Dimethicone | | | |
| | | Copolymer | | | |
| | PERMETHYL | Isododecane | 39.42 | 38.82 | 19.92 |
| | 99A | | | | |
| B | Pigments | | 3.63 | 3.63 | 3.63 |
| | Bentone gel | Disteardimonium | 10.00 | 10.00 | 20.00 |
| | | Hectorite/ | | | |
| | | isododecane/ | | | |
| | | propylene | | | |
| | | carbonate | | | |
| | | (10/87/3) | | | |
| | PERMETHYL | Isododecane | 8.45 | 8.45 | 8.45 |
| | 99A | | | | |
| C | FILLERS | | 8.50 | 8.50 | 8.50 |
| | AND PEARLS | | | | |
| | | TOTAL = | 100.00 | 100.00 | 100.00 |

The lip compositions of Examples 1-3 exhibited good wear, comfort, and a cushiony feel.

Preparation Procedure:

Phase A ingredients were combined together in a beaker, then the beaker transferred to a 100° C. oil bath and mixed with a propeller mixer until the solution was completely uniform.

Phase B ingredients were combined together into Phase A, then mixed about 5 minutes.

Phase C ingredients were added into Phase (A+B) solution and mixed for about 5 minutes.

The resulting fluid was transferred into individual packages.

Procedure of Preparing Phase B (Color Grind)

Phase B ingredients were combined together, then mixed by hand until the pigments were wet with oil.

The mixture was transferred to the Disconti-Mill and milled for 1 hour until the color grind became homogeneous.

The color grind phase B was transferred into the container for use in the procedure above.

### Examples 4 and 5

### Lip Compositions

| | | | Ex. 4 | Ex. 5 |
|---|---|---|---|---|
| PHASE | Trade Name | INCI Name | % w/w | % w/w |
| A | KP550 | Acrylates/ | 35.00 | 21.10 |
| | | Dimethicone | | |
| | | Copolymer | | |
| | | (40% solids in | | |
| | | Isododecane) | | |
| | DOW | Polypropyl- | 17.50 | 24.45 |
| | CORNING | silsesquioxane | | |
| | DC 670 | in Isododecane | | |
| | (80% solids) | | | |
| | PERMETHYL | Isododecane | 6.92 | 13.87 |
| | 99A | | | |
| B | Pigments | | 3.63 | 3.63 |
| | Bentone gel | Disteardimonium | 20.00 | 20.00 |
| | | Hectorite/ | | |
| | | Isododecane/ | | |
| | | Propylene | | |
| | | Carbonate | | |
| | | (10/87/3) | | |
| | PERMETHYL | Isododecane | 8.45 | 8.45 |
| | 99A | | | |
| C | Fillers/ | | 8.50 | 8.50 |
| | Pearls | | | |
| | | TOTAL = | 100.00 | 100.00 |

The lip compositions of Examples 4-5 exhibited good wear, comfort, and a cushiony feel.

Preparation Procedure:

Phase A ingredients were combined together in a beaker, then the beaker transferred to a 100° C. oil bath and mixed with a propeller mixer until the solution was completely uniform.

Phase B ingredients were combined together into Phase A, then mixed for about 5 minutes.

Phase C ingredients were added into Phase (A+B) solution and mixed for about 5 minutes.

The resulting fluid was transferred to individual packages.

Procedure of Preparing Phase B (Color Grind)

Phase B ingredients were combined together, then mixed by hand until the pigments were wet with oil.

The mixture was transferred to the Disconti-Mill and milled for 1 hour until the color grind became homogeneous.

The color grind phase B was transferred into the container for use in the procedure above.

### Example 6

### Compact Foundation

| PHASE | Trade Name | INCI Name | % w/w |
|---|---|---|---|
| A1 | DC-246 | Cyclohexasiloxane | 10.59 |
| A1 | DC-245 | Cyclopentasiloxane | 4.00 |
| A1 | Abil EM 90 | Cetyl PEG/PPG-10/1 | 2.40 |
| | | Dimethicone | |
| A1 | DC 2-8179 | Nylon-611/ | 1.25 |
| | | Dimethicone Copolymer | |
| A2 | Performal 400 | Polyethylene | 4.00 |
| A2 | Polywax 500 | Polyethylene | 1.00 |
| B1 | | Pigments | 13.46 |
| B1 | DC-246 | Cyclohexasiloxane | 10.00 |
| B2 | DC-670 | Polypropylsilsesquioxane | 12.00 |
| | | in Cyclomethicone | |
| | | (50% solids) | |
| B2 | KSG-710 | Polyglycerin Modified | 5.00 |
| | | Branched Silicone | |
| | | Crosspolymer | |
| B3 | Sunsphere Silica | Silica | 1.00 |
| B3 | Luxsil | Calcium Sodium | 4.00 |
| | | Borosilicate | |
| C1 | Water | Deionized Water | 30.00 |
| C1 | Hydrolite-5 | Pentylene Glycol | 0.50 |
| C1 | Preservatives | | 0.40 |
| C2 | Preservatives | | 0.40 |
| | | Total | 100.00 |

The foundation had excellent texture and feel and was transfer resistant.

Preparation Procedure

All mixing done in a beaker was performed at about 85° C. (in a heated water bath) at high shear.

Oil Phase (Main Beaker)

Phase A1 ingredients were mixed in main beaker until the Nylon-611/Dimethicone Copolymer was fully dissolved in silicone oils.

Phase A2 (waxes) were added with mixing into the main beaker until they were fully dissolved in phase A1.

Phase B1, pigment grind, was added to the main beaker while mixing was continued.

Phase B2 (DC670 and KSG-710) was added while mixing was continued.

Phase B3 was added to the mixture while mixing was continued.

Water Phase (Prepared at Same Time as Oil Phase at About 85° C. in a Heated Water Bath).

Phase C1 ingredients were combined in a separate beaker and mixed while heated.

Once the temperature reached about 85° C., Phase C2 was added and mixing continued for about 10 minutes.

Once Phases A and B were fully mixed (about 15 minutes), the hot water phase was added to the oil phase in the main beaker at high shear.

Mixing was continued for about 15 to 20 minutes at high shear.

The resulting mixture was hot poured into compacts and allowed to cool down.

### Examples 7 and 8

### Mascara Formulations

| | | Ex. 7 | Ex. 8 |
|---|---|---|---|
| Trade Name | INCI Name | % w/w | % w/w |
| | Isododecane | 31.00 | 31.25 |
| DC 2-8179 | Nylon-611/Dimethicone | 0 | 3.00 |
| | Copolymer | | |
| | Paraffin | 12.00 | 12.00 |
| | Beeswax | 3.00 | 3.00 |
| | Carnauba Wax | 3.00 | 3.00 |
| | Black Iron Oxide pigments | 10.00 | 10.00 |
| DC 670 | Polypropylsilsesquioxane in | 37.00 | 33.75 |
| | isododecane (80% solids) | | |
| Bentone | Disteardimonium Hectorite | 3.00 | 3.00 |
| | Propylene Carbonate | 1.00 | 1.00 |
| | TOTAL | 100.00 | 100.00 |

Example 8 (DC670 and Nylon-611/Dimethicone Copolymer) exhibited a smooth texture. Upon application on lashes, it had very good color intensity and exhibited lengthening, separation, volumizing of the lashes upon multiple applications, without balling. Example 7 had a grainy appearance and exhibited some balling upon application.

Preparation Procedure

In the main kettle, the isododecane was heated to about 65-75° C.

While mixing under heavy shear, the Nylon-611/Dimethicone Copolymer, and the waxes (Paraffin, Beeswax and Carnauba Wax) were added.

Mixing was continued until the mixture was homogeneous.

The pigments were added and mixed under heavy shear for about 1 hour.

Heating was stopped and the polypropylsilsesquioxane and bentone were added.

Once the temperature reached about 60-65° C., the propylene carbonate was added while mixing was continued.

Once all the ingredients have been well mixed, the samples were filled in mascara tubes.

What is claimed is:

1. A cosmetic composition comprising:
(a) at least one polypropylsilsesquioxane film forming resin;
(b) at least one polymer chosen from a polyorganosiloxane copolymer comprising nylon-611/dimethicone copolymer;
(c) at least one solvent; and
(d) optionally, at least one colorant.

2. The composition of claim 1, wherein said at least one polypropylsilsesquioxane is present in an amount of from about 0.5 to about 80% by weight, based on the weight of the composition.

3. The composition of claim 1, wherein said at least one polypropylsilsesquioxane is present in an amount of from about to about 40% by weight, based on the weight of the composition.

4. The composition of claim 1, wherein said at least one polypropylsilsesquioxane is present in an amount of from about to about 30% by weight, based on the weight of the composition.

5. The composition of claim 1, wherein said nylon-611/dimethicone copolymer is present in the composition in an amount of from about 0.5 to about 30% by weight, based on the weight of the composition.

6. The composition of claim 1, wherein said nylon-611/dimethicone copolymer is present in the composition in an amount of from about 1 to about 10% by weight, based on the weight of the composition.

7. The composition of claim 1, wherein said at least one solvent is present in an amount of from about 10 to about 90% by weight, based on the weight of the composition.

8. The composition of claim 1, wherein said at least one colorant is present in an amount of from about 0.1 to about 50% by weight, based on the weight of the composition.

9. The composition of claim 1, wherein said at least one colorant is present in an amount of from about 1 to about 20% by weight, based on the weight of the composition.

10. The composition of claim 1, wherein the composition is a lipstick.

11. The composition of claim 1, wherein the composition is a foundation.

12. The composition of claim 1, wherein the composition is a mascara.

13. The composition of claim 1, wherein the composition is an eye shadow.

14. A process for treating a keratinous material comprising:
(a) providing a keratinous material;
(b) providing a cosmetic composition containing:
(i) at least one polypropylsilsesquioxane film forming resin;
(ii) at least one polymer chosen from a polyorganosiloxane copolymer comprising nylon-611/dimethicone copolymer;
(iii) at least one solvent;
(iv) and optionally, at least one colorant; and
(c) applying the cosmetic composition onto the keratinous material.

15. The process of claim 14, wherein said at least one polypropylsilsesquioxane is present in an amount of from about 0.5 to about 80% by weight, based on the weight of the composition.

16. The process of claim 14, wherein said at least one polypropylsilsesquioxane is present in an amount of from about to about 40% by weight, based on the weight of the composition.

17. The process of claim 14, wherein said at least one polypropylsilsesquioxane is present in an amount of from about to about 30% by weight, based on the weight of the composition.

18. The process of claim 14, wherein said nylon-611/dimethicone copolymer is present in the composition in an amount of from about 0.5 to about 30% by weight, based on the weight of the composition.

19. The process of claim 14, wherein said nylon-611/dimethicone copolymer is present in the composition in an amount of from about 1 to about 10% by weight, based on the weight of the composition.

20. The process of claim 14, wherein said at least one solvent is present in an amount of from about 10 to about 90% by weight, based on the weight of the composition.

21. The process of claim 14, wherein said at least one solvent is present in an amount of from about 40 to about 70% by weight, based on the weight of the composition.

22. The process of claim 14, wherein said at least one colorant is present in an amount of from about 0.1 to about 50% by weight, based on the weight of the composition.

23. The process of claim 14, wherein said at least one colorant is present in an amount of from about 1 to about 20% by weight, based on the weight of the composition.

24. The process of claim 14, wherein the composition is a lipstick.

25. The process of claim 14, wherein the composition is a foundation.

26. The process of claim 14, wherein the composition is a mascara.

27. The process of claim 14, wherein the composition is an eye shadow.
